(19) United States
(12) Patent Application Publication
(10) Pub. No.: US 2014/0081333 A1
(43) Pub. Date: Mar. 20, 2014

(54) Dynamic stabilization medical implant assembles and methods

(71) Applicant: Roger P. Jackson, Prairie Village, KS (US)

(72) Inventor: Roger P. Jackson, Prairie Village, KS (US)

(21) Appl. No.: 13/573,474

(22) Filed: Sep. 17, 2012

(51) Int. Cl. A61B 17/70; A61B 17/88; A61B 17/86

(52) U.S. Cl. 606/257; 606/305; 606/267; 606/279

(57) ABSTRACT

Bone screw assemblies include longitudinal connecting members that provide for dynamic stabilization, some including non-uniform portions that are configured to flex, contract or expand. Composite longitudinal connecting members include longitudinal segments made from different materials having different flexibilities. Polyaxial bone screw assemblies include change-out receivers for cooperating with replacement longitudinal connecting members having a different flexibility. Bone screw shanks for cooperating with one or more open receivers include treatment or coating to provide biologically active interface with bone.

### CROSS-REFERENCE TO RELATED APPLICATIONS

This application is a divisional of U.S. patent application Ser. No. 11/509,386 that claimed the benefit of U.S. Provisional Application No. 60/722,300 filed Sep. 30, 2005. U.S. patent application Ser. No. 11/509,386 was also a continuation-in-part of U.S. patent application Ser. No. 10/958,743 filed Oct. 5, 2004, which is a continuation-in-part of U.S. patent application Ser. No. 10/409,935 filed Apr. 9, 2003, now U.S. Pat. No. 6,964,666. U.S. patent application Ser. No. 11/509,386 was also a continuation-in-part of U.S. patent application Ser. No. 10/818,555, filed Apr. 5, 2004, which is a continuation of U.S. patent application Ser. No. 10/464,633 filed Jun. 18, 2003, now U.S. Pat. No. 6,716,214. U.S. patent application Ser. No. 11/509,386 was also a continuation-in-part of U.S. patent application Ser. No. 10/996,349 filed Nov. 23, 2004.

### BACKGROUND OF THE INVENTION

The present invention relates to apparatuses and methods for use in performing spinal surgery and, in particular, to structural members for use in spinal surgery including dynamic stabilization longitudinal connecting members and cooperating polyaxial bone screw assemblies providing protected motion in a non-fusion procedure. Certain flexible elongate connecting members or rods used in methods according to the invention have a substantially uniform shape, while other longitudinal connecting members according to the invention have flexible non-uniform portions with varied cross-section that preserve spinal motion, providing for flexure and/or compression and extension, in a dynamic stabilization method and structure. Bone screws according to the invention have a receiver for capturing and clamping a longitudinal connecting member that can swivel about a shank of the bone screw, allowing the receiver to be positioned in any of a number of angular configurations relative to the shank. Receivers according to the invention also include a change out feature, allowing for removal of a flexible longitudinal connecting member and cooperating receiver without removing the threaded shank that has been implanted into bone, and then replacing the receiver with a second receiver for accommodating a more rigid longitudinal connecting member of a different size.

Many spinal surgery procedures require securing various implants to bone and especially to vertebrae along the spine. For example, elongate longitudinal connecting members are often required that extend along a portion of the spine to provide support to vertebrae that have been damaged or weakened due to injury, disease or the like. Such longitudinal connecting members must be supported by certain vertebra and support other vertebra. The most common mechanism for providing such structure is to implant bone screws into certain bones which then in turn support the longitudinal connecting member or are supported by the longitudinal connecting member. Bone screws typically have a shank that is threaded and adapted to be implanted into a vertebral body of a vertebrae. Such bone screws also include a receiver designed to extend beyond the vertebrae and include a channel for receiving a longitudinal connecting member or other elongate member. The receiver may be open, swiveling with respect to the shank, providing ease in placement of the longitudinal connecting member within the receiver channel prior to clamping of the longitudinal connecting member within the channel and locking the longitudinal connecting member with respect to the receiver and the shank in a particular desired angle with respect to the shank, utilizing a closure member that also is inserted in the receiver channel.

Historically, it has been common to fuse adjacent vertebrae that are placed in fixed relation by the installation therealong of bone screws or other bone anchors and cooperating longitudinal connecting members or other elongate members. Fusion results in the permanent immobilization of one or more of the intervertebral joints. Because the anchoring of bone screws, hooks and other types of anchors directly to a vertebra can result in significant forces being placed on the vertebra, and such forces may ultimately result in the loosening of the bone screw or other anchor from the vertebra; fusion allows for the growth and development of a bone counterpart to the longitudinal connecting member that can maintain the spine in the desired position even if the implants ultimately fail or are removed. Because fusion has been a desired component of spinal stabilization procedures, longitudinal connecting members have been designed that are of a material, size and shape to largely resist flexure, extension and compression, and thus substantially immobilize the portion of the spine that is to be fused. Thus, longitudinal connecting members are typically uniform along an entire length thereof, and usually made from a single or integral piece of material having a uniform diameter of a size to provide substantially rigid support.

Fusion, however, has some undesirable side effects. One apparent side effect is the immobilization of a portion of the spine. Furthermore, although fusion may result in a strengthened portion of the spine, it also has been linked to more rapid degeneration and even hyper mobility of spinal motion segments that are adjacent to the portion of the spine being fused, reducing or eliminating the ability of such spinal joints to move in a more normal relation to one another. In certain instances, fusion has also failed to provide pain relief.

An alternative to fusion and the use of rigid longitudinal connecting members or other rigid structure has been a “soft” stabilization approach in which a flexible C- or U-shaped member or coil is utilized as a spring member fixed between a pair of pedicle screws in an attempt to create, as much as possible, a normal loading pattern between the vertebrae, both in flexion and extension. Such devices allow for some natural movement or flex. However, such devices may be undesirable as they extend upwardly and outwardly from the bone screw or anchor, creating an implant with a profile much larger than those using a traditional cylindrical longitudinal connecting member. Larger profile implants are almost always undesirable for placement in a human body and may limit the working space afforded to the surgeon during an implant procedure.

Another concern that arises when more flexible structure is utilized in a spinal medical implant is that of adequate fatigue strength or endurance limit. The concept of strength may be defined as the highest stress a material can withstand before it completely fails to perform structurally. Typically, the concept of strength takes into account the influence of a force upon a cross-sectional area of a material that ultimately causes a material to fail. Specifically, fatigue strength has been defined as the repeated loading and unloading of a specific stress on a material structure until it fails. Fatigue strength can be tensile, compression, shear, bending, or a combination of these. The dynamic conditions associated with spinal movement therefore provide quite a challenge for the design of elongate structural members that exhibit an adequate fatigue strength to provide stabilization and protected motion of the spine, without fusion, and allow for some natural movement of the portion of the spine being reinforced by the elongate structural member.

### SUMMARY OF THE INVENTION

Dynamic medical implant assemblies and methods according to the invention include various longitudinal connecting members. One such member has first, second and third integral and substantially coaxial portions. The first and second portions are substantially uniform and are sized and shaped to be receivable in an open receiver of a bone attachment structure. The third portion is non-uniform and is disposed between the first and second portions. In one embodiment, the third portion includes first and second substantially parallel axially spaced sides and a plurality of curved strips, each curved strip being integral with both the first side and the second side at either end thereof. The third, non-uniform portion therefore being both compressible and expandable in an axial direction. The third portion is hollow and appears substantially spheroidal when in an extended orientation.

Another longitudinal connecting member of the invention includes first, second and third integral portions, the first portion being substantially uniform and having a first diameter, the second portion being substantially uniform and having a second diameter and the third portion being solid and disposed between the first and second portions. The first and second portions are illustrated herein as being cylindrical in form with equal diameters. The third portion has a first width defined by a first longitudinal cross-section and a second width defined by a second cross-section disposed perpendicular to the first longitudinal cross-section. In the illustrated embodiments, the first width of the non-uniform portion is larger than the diameters of the first and second portions and the second width is smaller than the diameters of the first and second portions.

A further longitudinal connecting member according to the invention has a substantially uniform cross section, but is divided longitudinally into at least first and second segments wherein the first segment is substantially more flexible in comparison to the second section. Each section may be sized and shaped to be received by at least a pair of bone anchors, allowing for dynamic stabilization along one portion of the spine and rigid stabilization along a second portion of the spine by a single longitudinal connecting member. Such longitudinal connecting members may have at least a portion of the first segment being constructed of material different from the second segment. Such longitudinal connecting members may include first and second segments that are both made from a solid material.

Dynamic medical implant assemblies according to the invention that provide dynamic, protected motion of the spine further include bone anchors, such as polyaxial bone screw assemblies, that may include bone screw shanks that are treated to provide for a roughened or textured surface, such as by plasma cleaning or coating. Furthermore, such treatment may include coating with a material such as hydroxyapatite. Such treatments and coatings provide for bone bonding and in certain cases a bioactive interface between the bone attachment structure and the vertebra.

Further apparatus and methods according to the invention include providing a first assembly for use with a flexible longitudinal connecting member or a longitudinal connecting member with flexible portions, and also providing replacement receivers for receiving longitudinal connecting members of different diameters, for example, for implanting at a later time when a more rigid assembly may be required. Specifically, polyaxial bone screw assemblies are described herein that include a first receiver for cooperating with a first longitudinal connecting member, such as flexible, dynamic stabilization connecting member and also a second receiver for cooperating with a second longitudinal connecting member having a different diameter and flexibility. Both the first and second receivers are attachable and detachable to a bone screw shank, both prior to implantation and after the shank is implanted in a vertebra. Such allows for a fusionless initial attachment of the bone screw shank, first receiver and dynamic stabilization connecting member to the spine. Thereafter, if needed, during a procedure in which the bone screw shank remains implanted, the first receiver may be replaced with the second receiver and the dynamic stabilization connecting member replaced with another longitudinal connecting member having a different flexibility and a different diameter, for example, a solid rod having a smaller diameter, but greater rigidity. In some instances, it may be desirable to replace a first longitudinal connecting member with a second, more flexible connecting member having a greater or lesser diameter than the first longitudinal member. In all such dynamic stabilization procedures that do not include fusion, one aspect of the invention is to provide bone screw shanks that have had surface treatment or coating to provide a biologically active interface with the bone or at least some component of bone bonding on or bone ingrowth into the bone screw shank.

### Objects and Advantages of the Invention

Therefore, it is an object of the present invention to overcome one or more of the problems with polyaxial bone screw assemblies described above. An object of the invention is to provide dynamic medical implant stabilization assemblies and methods for spinal surgery that include bone screws having an affinity to bone and further include connecting members and/or receiver members that may be removed and replaced to provide for the implantation of flexible, semi-rigid or rigid connecting members. Another object of the invention is to provide dynamic medical implant stabilization assemblies having longitudinal connecting members with portions having various configurations for providing flexible, dynamic stabilization. Additionally, it is an object of the invention to provide a lightweight, reduced volume, low profile polyaxial bone screw and longitudinal connecting member assembly. Furthermore, it is an object of the invention to provide apparatus and methods that are easy to use and especially adapted for the intended use thereof and wherein the tools are comparatively inexpensive to make and suitable for use.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an exploded perspective view of a polyaxial bone screw assembly according to the present invention having a shank with a capture structure at an end thereof, a first receiver, and a closure structure.

FIG. 2 is an enlarged and fragmentary view of the assembly of FIG. 1, showing the first receiver in cross-section, taken along the line 2-2 of FIG. 1, and illustrating the shank in front elevation prior to the insertion of the shank capture structure into the receiver according to a method of the invention.

FIG. 3 is a reduced and fragmentary cross-sectional view similar to FIG. 2 showing the shank capture structure being installed in the first receiver.

FIG. 4 is an enlarged and fragmentary cross-sectional view of the first receiver taken along the line 2-2 of FIG. 1 and the shank taken along the line 4-4 of FIG. 1, illustrating the shank capture structure fully installed in the receiver and swivelable therein.

FIG. 5 is a reduced and fragmentary cross-sectional view of the first receiver and attached shank of FIG. 4, and further showing the shank being implanted into a vertebra using a driving tool mounted on the shank capture structure, the driving tool shown in front elevation.

FIG. 6 is an enlarged and fragmentary cross-sectional view, similar to FIG. 5, also showing the driving tool in cross section.

FIG. 7 is a reduced and fragmentary cross-sectional view of the first receiver and vertebra, similar to FIG. 5, showing the shank in front elevation and showing a longitudinal connecting member, in cross-section, disposed in the receiver, and further illustrating the insertion of the closure structure of FIG. 1 using a driver, the closure structure and driver shown in front elevation.

FIG. 8 is a reduced and fragmentary front-elevational view of the assembly of FIG. 1, shown with a longitudinal connecting member in cross-section, the shank implanted in the vertebra and with the closure structure fully installed.

FIG. 9 is a reduced and exploded front elevational view of the shank of FIG. 1 shown implanted in a vertebra, shown with the first receiver and the longitudinal connecting member of FIG. 1 and also shown with a replacement receiver and a second, larger cooperating longitudinal connecting member.

FIG. 10 is a front elevational view similar to FIG. 9 showing the replacement receiver and the second longitudinal connecting member with the shank of FIG. 1.

FIG. 11 is a generally schematic side elevational view of a patient's spine, showing four tools manipulating four bone screws with receivers holding a flexible longitudinal connecting member.

FIG. 12 is a generally schematic side elevational view of a patient's spine, showing the four bone screw shanks of FIG. 11 now installed with replacement receivers and a larger replacement longitudinal connecting member.

FIG. 13 is an exploded perspective view of a second bone screw assembly according to the invention including a shank, a receiver and a retaining structure.

FIG. 14 is an exploded and side elevational view of the embodiment of FIG. 13 also shown with a longitudinal connecting member and further with a replacement receiver, retaining structure and longitudinal connecting member of greater rigidity.

FIG. 15 is a perspective view of a non-uniform longitudinal connecting member for use according to the invention.

FIG. 16 is a partial perspective view of a second embodiment of a non-uniform longitudinal connecting member.

FIG. 17 is a partial side elevational view of the longitudinal connecting member of FIG. 16.

FIG. 18 is a partial top plan view of the longitudinal connecting member of FIG. 16 shown with a schematic bone screw in phantom to illustrate orientation when implanted.

FIG. 19 is a cross-sectional view taken along the line 19-19 of FIG. 17.

FIG. 20 is a partial perspective view of a third embodiment of a non-uniform longitudinal connecting member.

FIG. 21 is a partial side elevational view of the longitudinal connecting member of FIG. 20.

FIG. 22 is a partial top plan view of the longitudinal connecting member of FIG. 20 shown with a schematic bone screw in phantom to illustrate orientation when implanted.

FIG. 23 is a partial perspective view of a fourth embodiment of a non-uniform longitudinal connecting member.

FIG. 24 is a partial side elevational view of the non-uniform longitudinal connecting member of FIG. 23 shown with a schematic bone screw in phantom to illustrate orientation when implanted.

FIG. 25 is a partial top plan view of the longitudinal connecting member of FIG. 23.

FIG. 26 is a partial perspective view of a fifth embodiment of a non-uniform longitudinal connecting member.

FIG. 27 is a partial side elevational view of the non-uniform longitudinal connecting member of FIG. 26.

FIG. 28 is a partial top plan view of the longitudinal connecting member of FIG. 26 shown with a schematic bone screw in phantom to illustrate orientation when implanted.

FIG. 29 is a partial perspective view of a sixth embodiment of a non-uniform longitudinal connecting member.

FIG. 30 is a partial side elevational view of the longitudinal connecting member of FIG. 29.

FIG. 31 is a partial top plan view of the non-uniform longitudinal connecting member of FIG. 29 shown with a schematic bone screw in phantom to illustrate orientation when implanted.

FIG. 32 is a cross-sectional view taken along the line 32-32 of FIG. 30.

FIG. 33 is a partial perspective view of a seventh embodiment of a non-uniform longitudinal connecting member.

FIG. 34 is a partial side elevational view of the longitudinal connecting member of FIG. 33.

FIG. 35 is a partial top plan view of the longitudinal connecting member of FIG. 33 shown with a schematic bone screw in phantom to illustrate orientation when implanted.

FIG. 36 is a partial perspective view of an eighth embodiment of a non-uniform longitudinal connecting member.

FIG. 37 is a partial side elevational view of the longitudinal connecting member of FIG. 36.

FIG. 38 is a cross-sectional view taken along the line 38-38 of FIG. 37.

FIG. 39 is a partial perspective view of a ninth embodiment of a non-uniform longitudinal connecting member.

FIG. 40 is a partial side elevational view of the longitudinal connecting member of FIG. 39.

FIG. 41 is a cross-sectional view taken along the line 41-41 of FIG. 40.

FIG. 42 is a partial perspective view of an tenth embodiment of a non-uniform longitudinal connecting member.

FIG. 43 is a partial side elevational view of the longitudinal connecting member of FIG. 42.

FIG. 44 is a cross-sectional view taken along the line 44-44 of FIG. 43.

FIG. 45 is a partial perspective view of an eleventh embodiment of a non-uniform longitudinal connecting member.

FIG. 46 is a perspective view of a mono-axial bone screw.

FIG. 47 is a partial front elevational view showing the longitudinal connecting member of FIG. 45 received in a receiver of the bone screw of FIG. 46 shown implanted in a vertebra.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

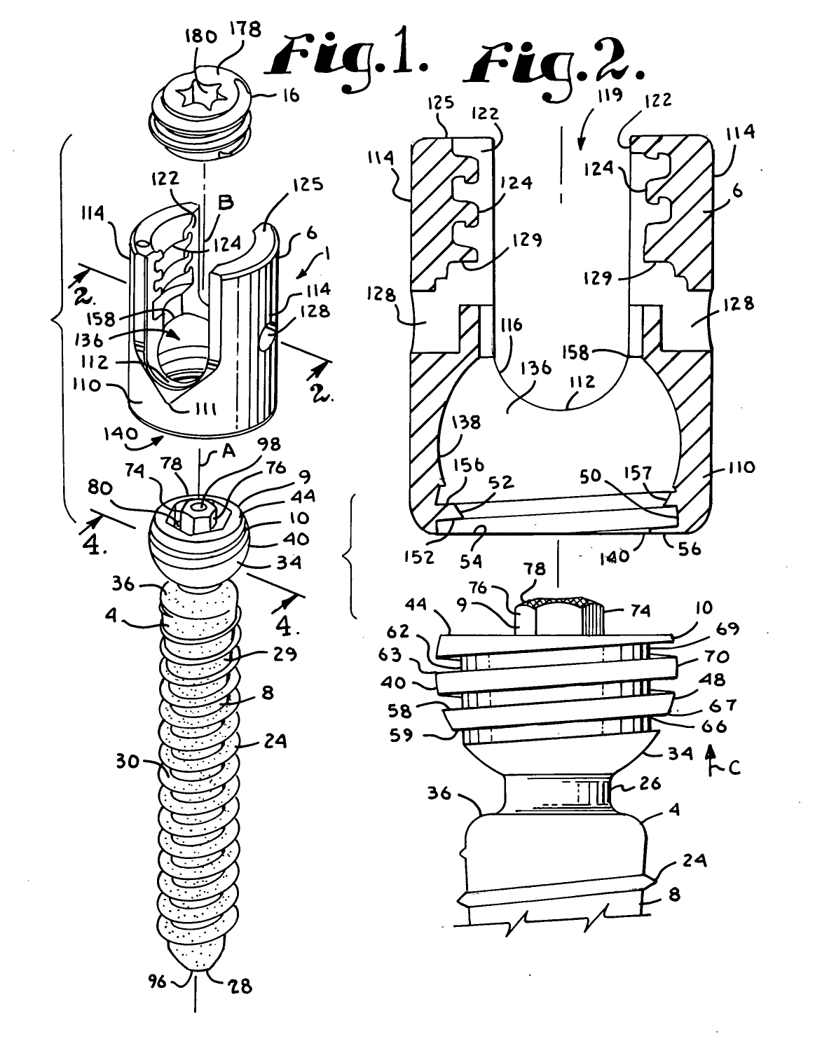

With reference to FIGS. 1-10, the reference number 1 generally represents a polyaxial bone screw apparatus or assembly according to the present invention. The assembly 1 includes a shank 4, a first receiver 6 and a second or replacement receiver 7. The shank 4 further includes a body 8 integral with an upper portion 9 having a capture structure 10. The shank 4 and the receiver 6 are often assembled prior to implantation of the shank body 8 into a vertebra 13, as seen in FIG. 5. However, in an alternative method, the shank body 8 may be first implanted in the vertebra 13, followed by joining the receiver 6 to the shank 4. Furthermore, as will be described in greater detail herein, the first receiver 6 may be removed from an implanted shank body 8 and the second receiver 7 joined to the shank 4 without the removal of the shank body 8 from the vertebra 13.

FIG. 1 further shows a closure structure 16 of the invention for biasing a longitudinal member such as a longitudinal connecting member 19 or a longitudinal connecting member 20 against the shank upper portion 9 which in turn biases the capture structure 10 into fixed frictional contact with the receiver 6 or 7, so as to fix the longitudinal connecting member 19 or 20 relative to the vertebra 13. The receiver 6 or 7 and the shank 4 cooperate in such a manner that the receiver 6 or 7 and the shank 4 can be secured at any of a plurality of angles, articulations or rotational alignments relative to one another and within a selected range of angles both from side to side and from front to rear, to enable flexible or articulated engagement of the receiver 6 or 7 with the shank 4 until both are locked or fixed relative to each other near an end of an implantation procedure.

With reference to FIGS. 1 and 2, the shank 4 is elongate, with the shank body 8 having a helically wound bone engaging thread 24 extending from near a neck 26 located adjacent to the capture structure 10 to near a tip 28 of the body 8 and projecting radially outwardly therefrom. To provide a biologically active interface with the bone, an outer surface 29 the shank body 8 that includes the thread 24 and extends between the neck 26 and the tip 28 is coated, perforated or otherwise treated 30. The treatment 30 may include, but is not limited to a plasma spray coating, a hydroxyapatite (HA) or tricalcium phosphate (TCP) coating, or other type of roughening, perforation or indentation in the surface 29, such as by sputtering, sand blasting or acid etching, that allows for bony ingrowth. Such treatments have been utilized, for example, on titanium dental implants in order to roughen the implant surface and to provide an enduring bond between confronting or interfacing surfaces of the dental implant and the host bone. Coating with hydroxyapatite, a bio-ceramic calcium phosphate coating, is desirable as hydroxyapatite is chemically similar to bone with respect to mineral content and has been identified as being bioactive and thus supportive of bone ingrowth in dental and maxillofacial applications.

During use, rotation of the body 8 utilizes the thread 24 for gripping and advancement in the bone and is implanted into the vertebra 13 leading with the tip 28 and driven down into the vertebra 13 with an installation or driving tool 31, so as to be implanted in the vertebra 13 to near the neck 26, as shown in FIG. 5 and as is described more fully in the paragraphs below.

The shank 4 has an elongate axis of rotation generally identified by the reference letter A. It is noted that any reference to the words top, bottom, up and down, and the like, in this application refers to the alignment shown in the various drawings, as well as the normal connotations applied to such devices, and is not intended to restrict positioning of the assembly 1 in actual use.

The neck 26 extends axially outwardly and upwardly from the shank body 8 to a base 34 of the capture structure 10. The neck 26 generally has a reduced radius as compared to an adjacent top 36 of the shank body 8. Further extending axially and outwardly from the neck 26 is the capture structure 10 that provides a connective or capture apparatus disposed at a distance from the body top 36 and thus at a distance from the vertebra 13 when the shank body 8 is implanted in the vertebra 13.

The capture structure 10 is configured for connecting the shank 4 to the receiver 6 or 7 and then capturing the shank 4 in the receiver 6 or 7. The capture structure 10 has an outer partially spherically shaped surface 40 extending from the base 34 to a top portion 44. The illustrated base 34 has a smooth surface, but it is foreseen that the base 34 may have a high-friction or roughened surface, such as a scored or knurled surface. Formed on an upper part 46 of the surface 40 is a helical guide and advancement structure 48. The guide and advancement structure 48 retains the substantially spherical outer shape of the surface 40 at a crest thereof, but may be otherwise described as a substantially square thread form, sized and shaped to mate with a cooperating guide and advancement structure 50 disposed on an inner surface 52 of the receiver 6 disposed adjacent to and defining an opening 54 of a lower end or bottom 56 of the receiver 6. Preferably, the guide and advancement structure 48 is relatively thick and heavy to give strength to the thread and prevent the thread from being easily bent or deformed when axial pressure is applied to the shank 4 to maintain the capture structure 10 in the receiver 6, as described further below. The second or replacement receiver 7 also includes an inner guide and advancement structure (not shown), substantially identical to the guide and advancement structure 50 for mating with the guide and advancement structure 48.

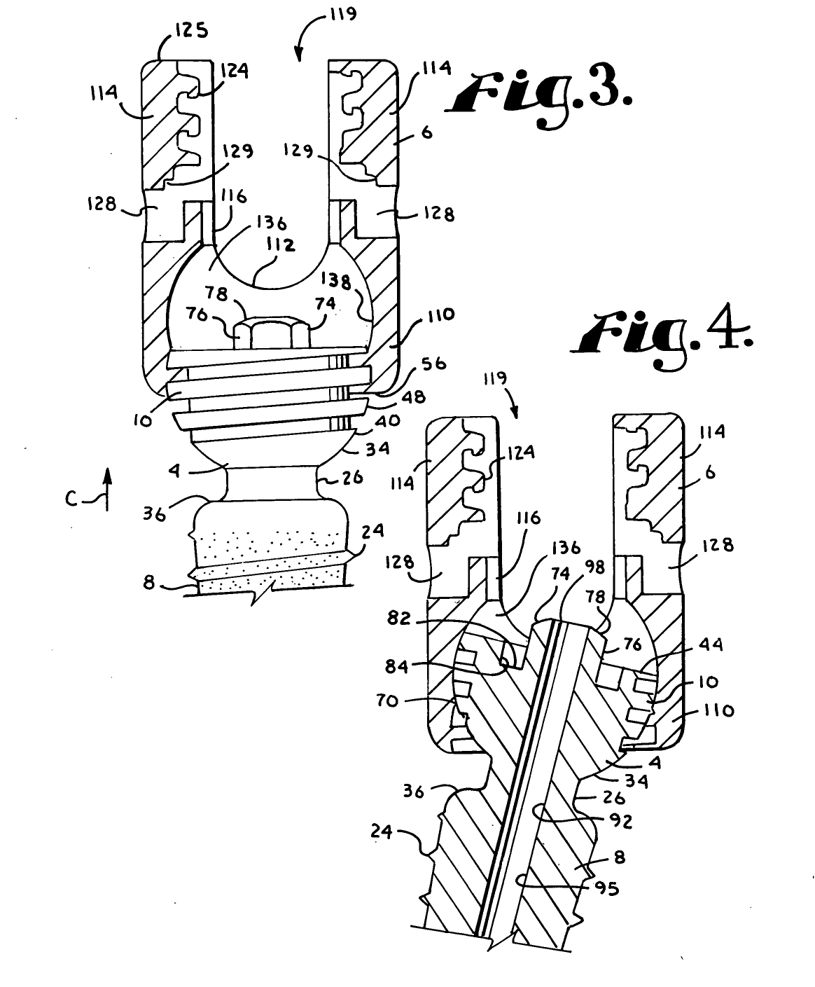

The guide and advancement structure 48 winds about the upper portion 46 in a generally helical pattern or configuration that is typical of threads and can have various pitches, be clockwise or counterclockwise advanced, or vary in most of the ways that conventional square threads vary. The guide and advancement structure 48 has a leading surface or flank 58 and a trailing surface or flank 59. As used herein, the terms leading and trailing refer to the direction of advancement of the capture structure 10 into the guide and advancement structure 50 of the receiver 6 aligning the axis A of the shank 4 with an elongate axis of rotation B of the receiver 6 and directing the capture structure 10 toward the receiver 6, as shown by the straight arrow C illustrated in FIGS. 2 and 3.

The leading surface 58 has an inner edge 62 and an outer edge 63. The trailing surface 59 has an inner edge 66 and an outer edge 67. As is typical of square threads, a root surface 69 between the inner edges 62 and 66 is parallel to the axis of rotation A and has an axial length that remains substantially constant throughout the threadform. Likewise, an axial distance between the outer edges 63 and 67 remains substantially constant, while the size of a crest or connecting surface 70 between the edges 63 and 67 varies, due to the spherical form of the crest surface 70. As can be seen, for example, in FIG. 4, the root surface 69 is disposed substantially perpendicular to the leading surface 58 and the trailing surface 59.

Although the substantially square threadform 48 is described herein, it is foreseen that other thread types, such as V-threads, inverted thread types, such as inverted buttress threads, other thread-like or non-thread-like guide and advancement structures, such as flange form helically wound advancement structures may be utilized according to the invention.

Advancement of the capture structure 10 into the receiver 6 is accomplished by rotating the shank 4 in a counterclockwise direction about the axes A and B and into the receiver 6 as illustrated in FIG. 3. As will be described more fully below, the connecting crest surface 70 is a loading surface after the capture structure 10 is fully disposed in the receiver 6. Also as will be described in more detail below, although discontinuous, the spherical surface 70 has an outer radius that is approximately equal to a radius of an inner seating surface of the receiver 6 or 7, allowing for slidable mating contact between the surface 70 and an inner seating surface of the receiver 6 or 7.

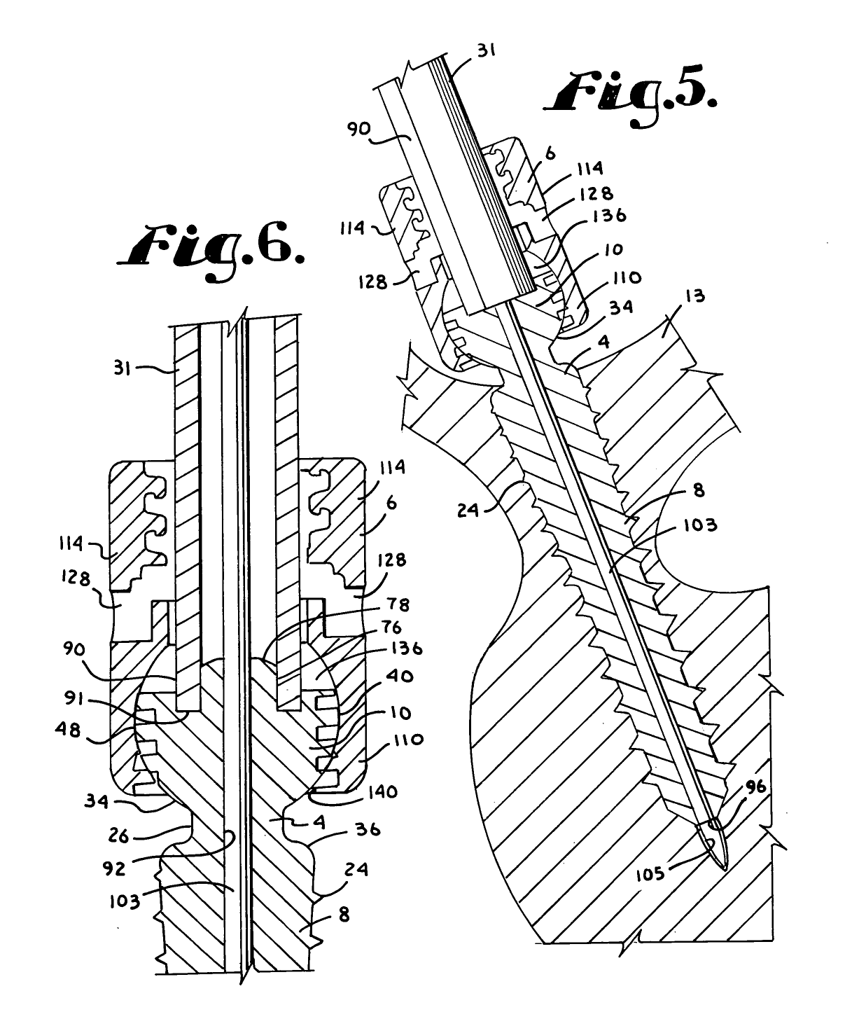

In the embodiment shown, the shank 4 further includes a longitudinal connecting member and tool engagement structure 74 projecting upwardly from the top portion 44 of the capture structure 10. The tool engagement structure 74 has a hexagonally shaped head 76 with a substantially domed top 78. The structure 74 is coaxial with both the threaded shank body 8 and the capture structure 10. The head 76 is sized and shaped for engagement with the driving tool 31 shown in FIGS. 5 and 6 that includes a driving and mating structure in the form of a socket. The tool 31 is configured to fit about the head 76 so as to form a socket and mating projection for both operably driving and rotating the shank body 8 into the vertebra 13.

In the embodiment shown, to provide further mechanical advantage during installation of the shank 4 into the vertebra 13, the capture structure 10 includes a counter-sunk portion 80 formed in the top 44, the portion 80 adjacent to and surrounding the head 76. The portion 80 includes a planar seating surface 82 disposed perpendicular to the axis A and spaced from the top portion 44. Contiguous to both the surface 82 and the top 44 are faces 84 that are disposed parallel to the axis A and thus are substantially perpendicular to the surface 82. The faces 84 form a hex-shaped outer periphery of the counter-sunk portion 80. The tool 31 includes an outer surface portion 90 sized and shaped to mate with the bottom and both side walls of the counter-sunk portion 80, such that a bottom 91 of the tool 31 seats on the surface 82 and the outer surface portion 90 is adjacent to and engaging the faces 84 when the tool 31 is disposed about and engaging with the hexagonally shaped head 76.

The domed top end surface 78 of the shank 4 is preferably convex, curved or dome-shaped as shown in the drawings, for positive engagement with the longitudinal connecting member 19 when the bone screw assembly 1 is assembled, as shown in FIGS. 8 and 10, and in any alignment of the shank 4 relative to the receiver 6 or 7. While not required for the practice of the invention, in the embodiment shown in the drawings, the top end surface 78 is scored or knurled to further increase frictional engagement between the surface 78 and the longitudinal connecting member 19. It is foreseen that in certain embodiments, the surface 78 may be smooth. The dome 78 may be radiused so that the dome 78 engages the longitudinal connecting member 19 slightly above a longitudinal connecting member receiving channel in the receiver 6 or 7, even as the receiver 6 or 7 is swivelled relative to the shank 4 so that pressure is always exerted on the dome surface 78 by the longitudinal connecting member 19 when the assembly 1 is fully assembled. It is foreseen that in other embodiments the dome 78 can have other shapes which may include off-axis apertures for driving the shank with a mating tool. The dome can also involve one or more arched sections with flat external surfaces which can mate with a driving tool.

The shank 4 shown in the drawings is cannulated, having a small central bore 92 extending an entire length of the shank 4 along the axis A. The bore 92 is defined by an inner substantially cylindrical wall 95 of the shank 4 and has a first circular opening 96 at the shank tip 28 and a second circular opening 98 at the top domed surface 78. The bore 92 is coaxial with the threaded body 8 and the capture structure 10. The bore 92 provides a passage through the shank 4 interior for a guide pin or length of wire 103 inserted into a small pre-drilled bore 105 in the vertebra 13 prior to the insertion of the shank body 8, the pin 103 providing a guide for insertion of the shank body 8 into the vertebra 13.

The receiver 6 is partially cylindrical in external profile and includes a base portion 110 extending from the end 56 to a V-shaped surface 111 disposed at a periphery of a longitudinal connecting member seating surface 112 and extending radially outwardly and downwardly therefrom. The base 110 is integral with a pair of upstanding and spaced arms 114. The surface 112 and the arms 114 forming a U-shaped channel 116 between the arms 114 and having an upper opening 119. The lower surface 110 defining the channel 116 preferably has substantially the same radius as the longitudinal connecting member 19. In operation, the longitudinal connecting member 19 preferably is located just above the channel lower surface 112, as shown in FIG. 8.

Each of the arms 114 has an interior surface 122 that defines an inner cylindrical profile and includes a discontinuous helically wound guide and advancement structure 124 beginning at a top 125 of the receiver 6 and extending downwardly therefrom. The guide and advancement structure 124 is a partial helically wound flange-form configured to mate under rotation about the axis B with a similar structure disposed on the closure structure 16, as described more fully below. However, it is foreseen that the guide and advancement structure 124 could alternatively be a V-shaped thread, a buttress thread, a square thread, a reverse angle thread or other thread-like or non-thread-like helically wound guide and advancement structure for operably guiding under rotation and advancing the closure structure 16 between the arms 114, as well as eventual torquing when the closure structure 16 abuts against the longitudinal connecting member 19.

The receiver 6 includes external apertures or grip bores 128 disposed on each of the arms 114 for positive engagement by holding tools to facilitate secure gripping of the receiver 6 during assembly of the receiver 6 with the shank 4. Furthermore, the grip bores 128 may be utilized to hold the receiver 6 during the implantation of the shank body 8 into the vertebra 13. The bores 128 are centrally located on the respective arms 114 and communicate with upwardly projecting hidden recesses 129 to further aid in securely holding the receiver 6, for example, to an end guide or holding tool 130 or an intermediate guide or holding tool 131 illustrated in FIG. 11, the guide tools 130 and 131 having structure (not shown) for communicating both with the bores 128 and recesses 129. The guide tools 130 and 131 have channels or slots (not shown) for alignment with the U-shaped channel 116 of the receiver 6 and sized and shaped to receive a longitudinal connecting member 19 or other elongate structure therethrough. The guide tools 130 and 131 are preferably further equipped with elongate channels extending along lengths thereof for the placement of closure structures 16 and other tools therein, utilized to press and lock a longitudinal connecting member 19 or other elongate structure within the receiver 6. It is foreseen that the bores 128 and recesses 129 may be configured to be of a variety of sizes, shapes and locations along outer surfaces of the arms 114 for cooperation with one or more holding tools.

Communicating with the U-shaped channel 116 of the receiver 6 is a chamber or cavity 136 substantially defined by a partially spherical inner surface 138 that is disposed primarily in the base portion 110 of the head beneath the interior cylindrical surface 122 of the arms 112 and 114 and extending into the inner surface 52 that is further defined by the guide and advancement structure 50. The cavity 136 communicates with both the U-shaped channel 116 and a bore 140 that also is defined by the guide and advancement structure 50, that in turn communicates with the opening 54 at the bottom 56 of the receiver 6.

The guide and advancement structure 50 includes a leading surface 152 and a trailing surface 156. Similar to what is described herein with respect to the guide and advancement structure 48 of the capture structure 10, the guide and advancement structure 50 is preferably of a square thread type as such structure provides strength and stability to the assembly 1, with the leading surface 152 and the trailing surface 156 being substantially parallel. A crest surface 157 spanning between the leading surface 152 and the trailing surface 156 is curvate, having a radius the same or substantially similar to a radius of the cavity spherical wall 138. As with the guide and advancement structure 48, it is foreseen that other types of threaded and non-threaded helical structures may be utilized in accordance with the present invention for the receiver 6.

A juncture of the interior surface 122 and the cavity inner surface 138 forms an opening or neck 158 that has a radius extending from the Axis B that is smaller than a radius extending from the Axis B to the inner surface 138. Also, a radius from the lower opening 54 to the Axis B is smaller than the radius extending from the Axis B to the inner surface 138 and the inner surface portion 52 defining the guide and advancement structure 50. Thus, the cavity or chamber 136 is substantially spherical, widening and opening outwardly and then inwardly in a direction toward the lower opening 54. However, it is foreseen that other shapes, such as a cone or conical shape, may be utilized for a head inner cavity according to the invention. Also, a cylindrical head inner cavity with a retainer ring located approximate the lower opening 54 could be utilized according to the invention.

After the guide and advancement structure 48 of the capture structure 10 is mated and rotated to a position within the cavity 136 and further upwardly and axially into non-engagement beyond the trailing surface 156 of the guide and advancement structure 50, the capture structure 10 is rotatable or swingable within the cavity 136 until later frictionally locked in place, and cannot be removed from the receiver 6 through the upper neck 158 or through the lower bore 140 without reversing the assembly process with the components in axial alignment. As shown in FIG. 4, the capture structure 10 is held within the cavity 136 from above by the partially spherical surface 138 and from below by the threaded inner surface 52. Stated in another way, the thick strong threadform 50 having the curvate surface 157 of the receiver 6 disposed along the surface 52, and the slidingly mated curvate surface 70 of the thick strong threadform 48 of the capture structure 10, prevent the capture structure 10 from being pushed or pulled from the chamber 136, unless the capture structure 10 is rotated and unscrewed therefrom again through the bore 140 in axial alignment. If there is no pressure from above, the cavity or chamber 136 allows the structure 10 to freely rotate in the chamber 136 to a position or orientation desired by a surgeon. In this manner, the receiver 6 is able to swivel or swing about the shank 4 until subsequently locked in place.

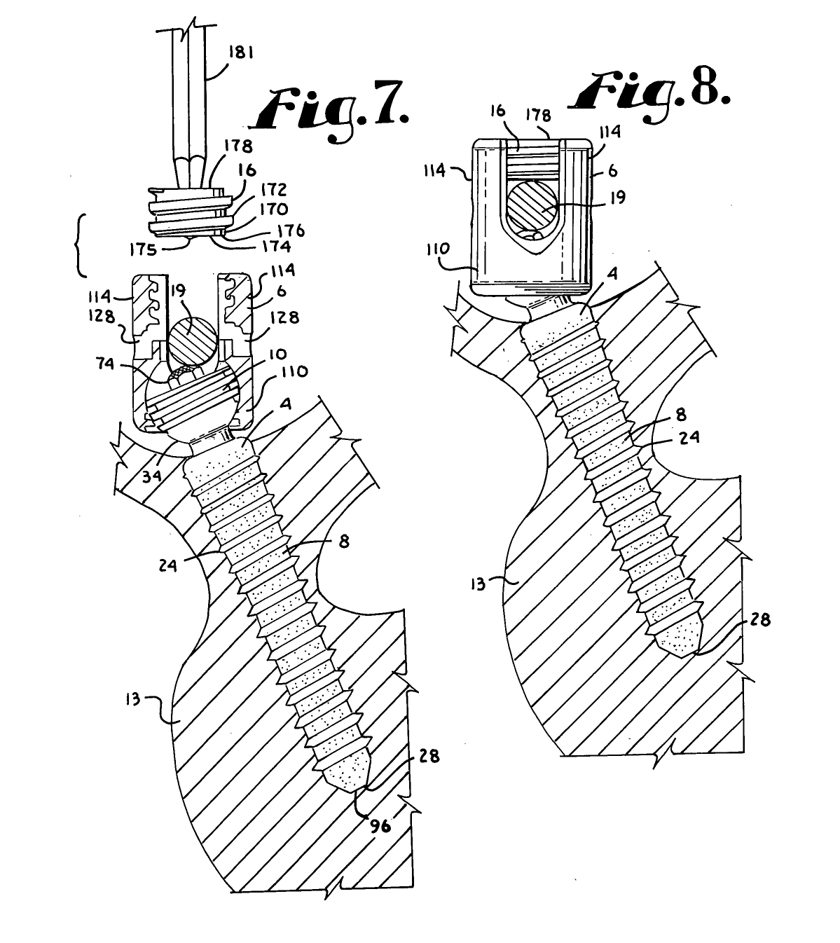

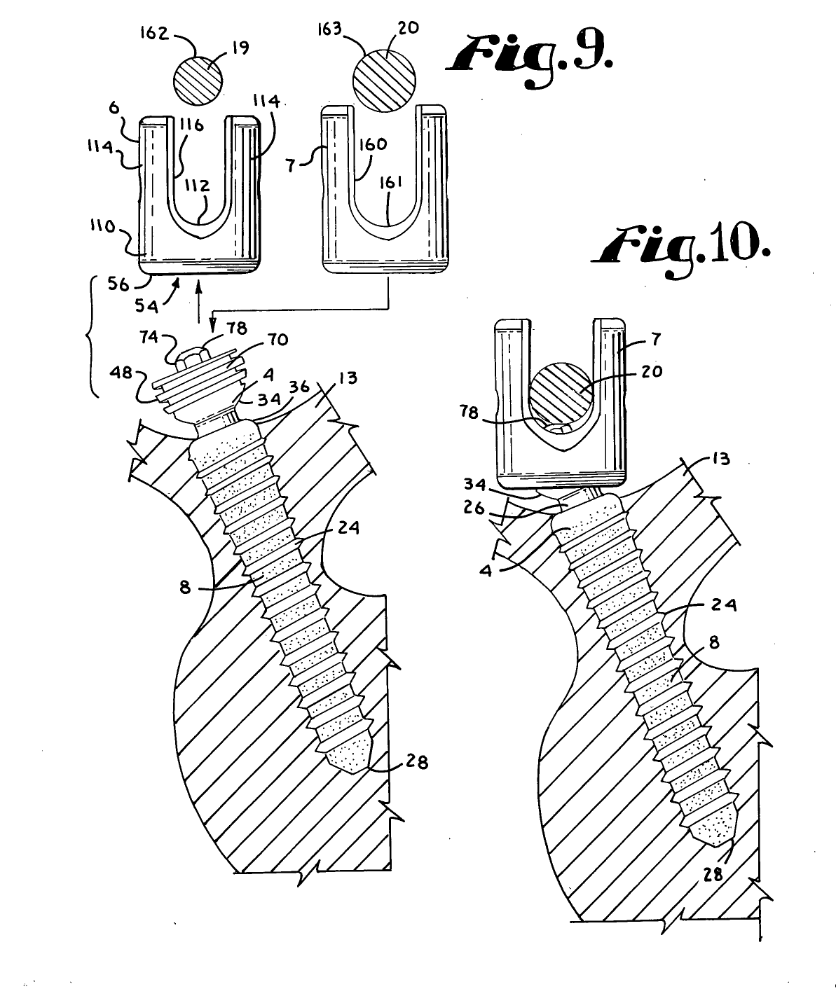

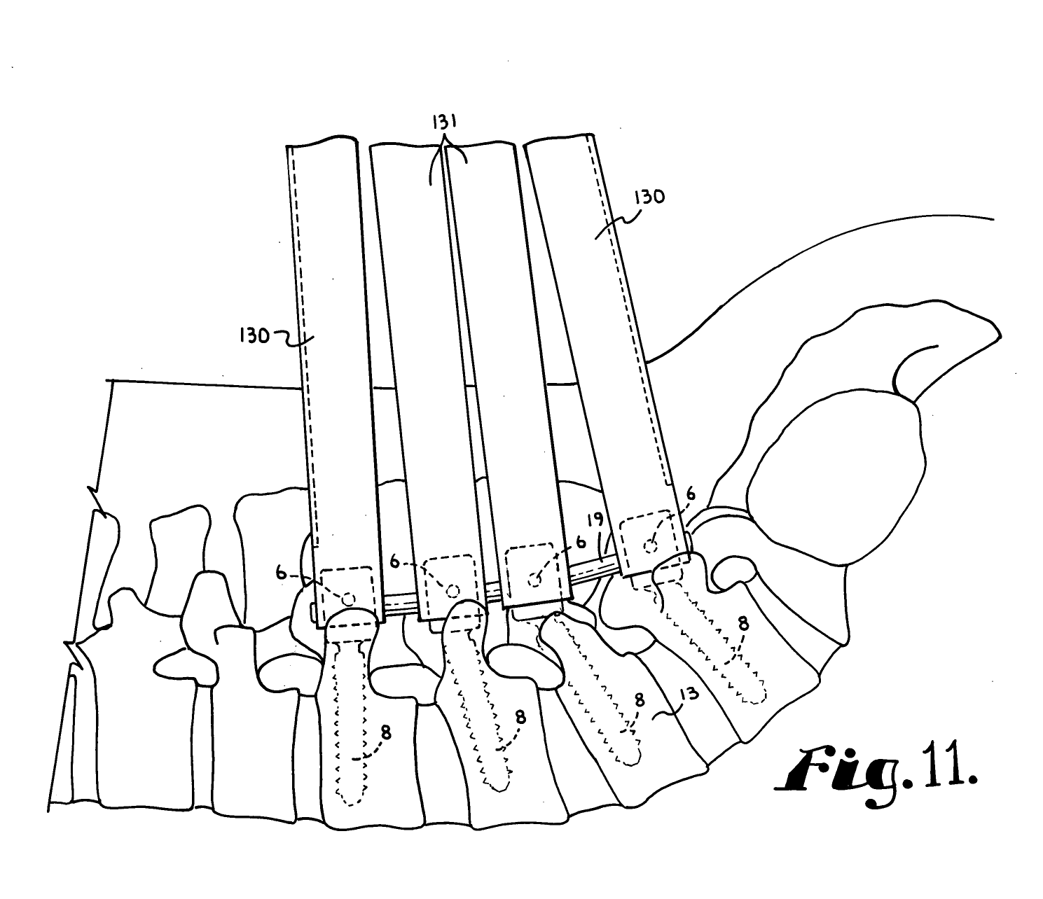

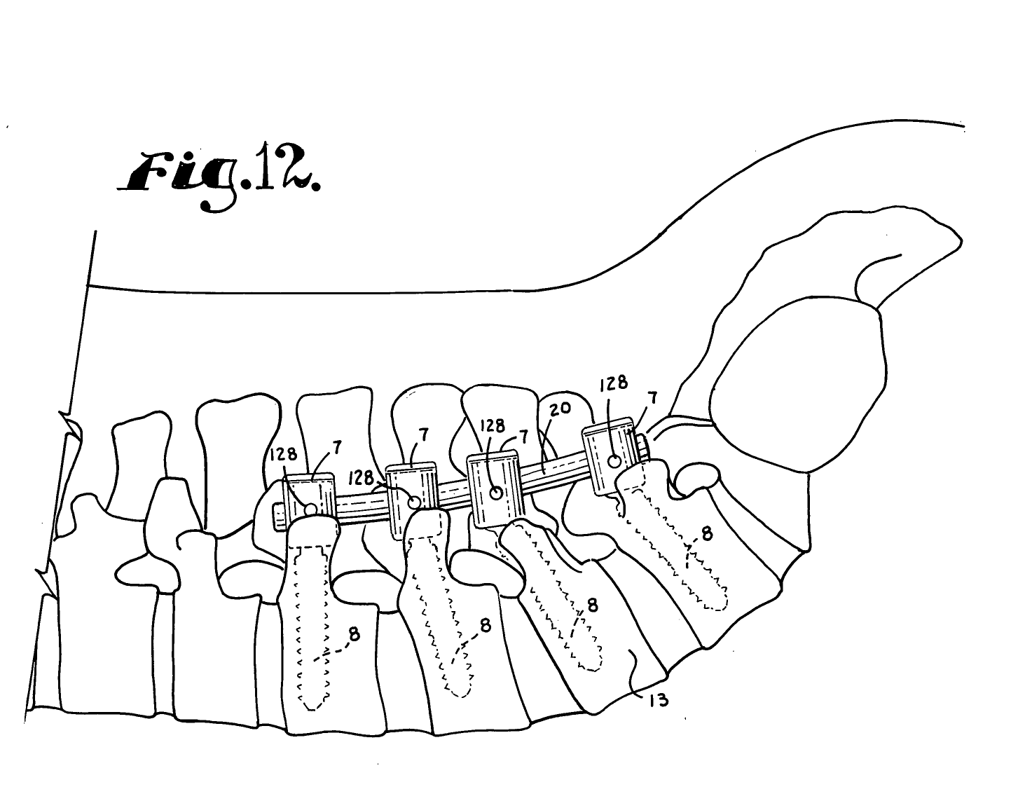

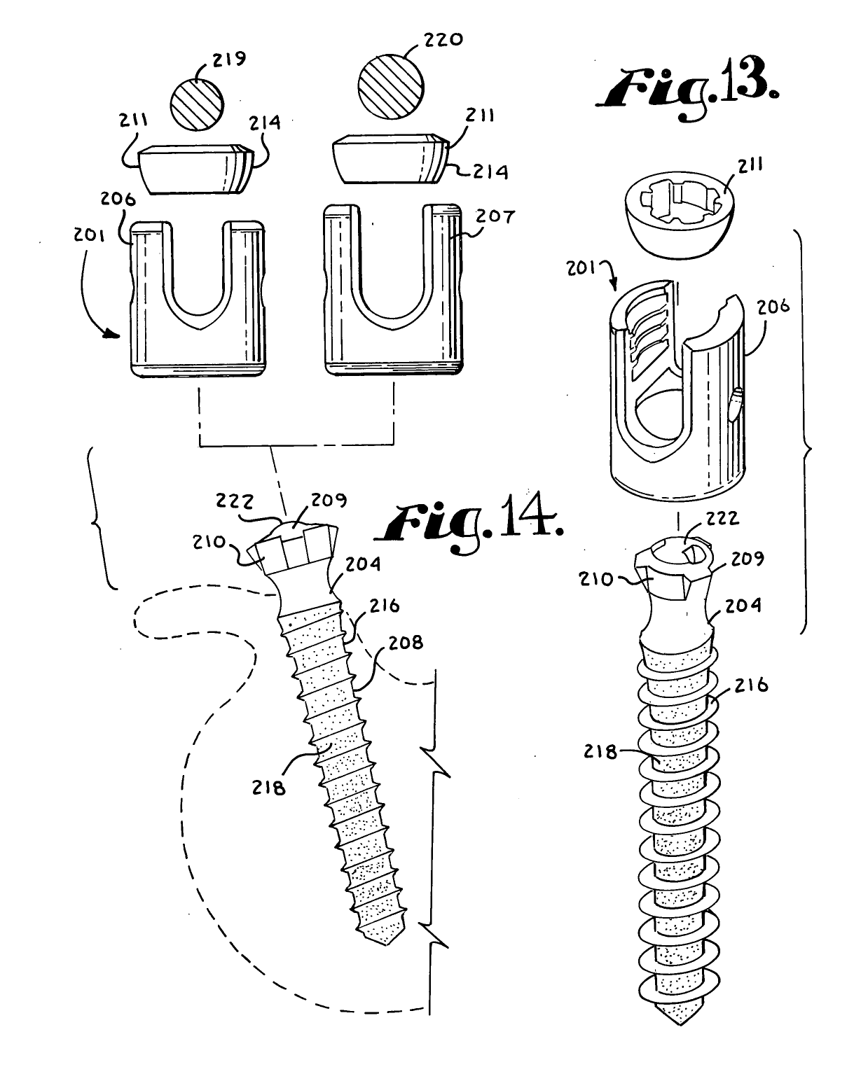

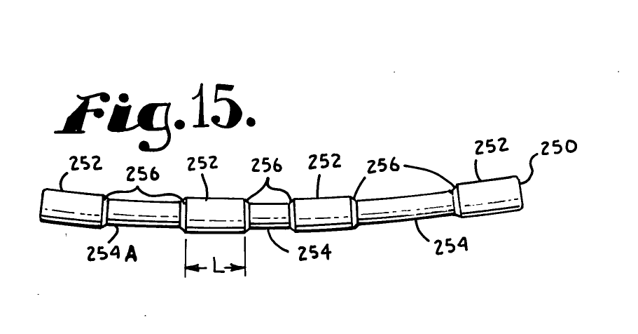

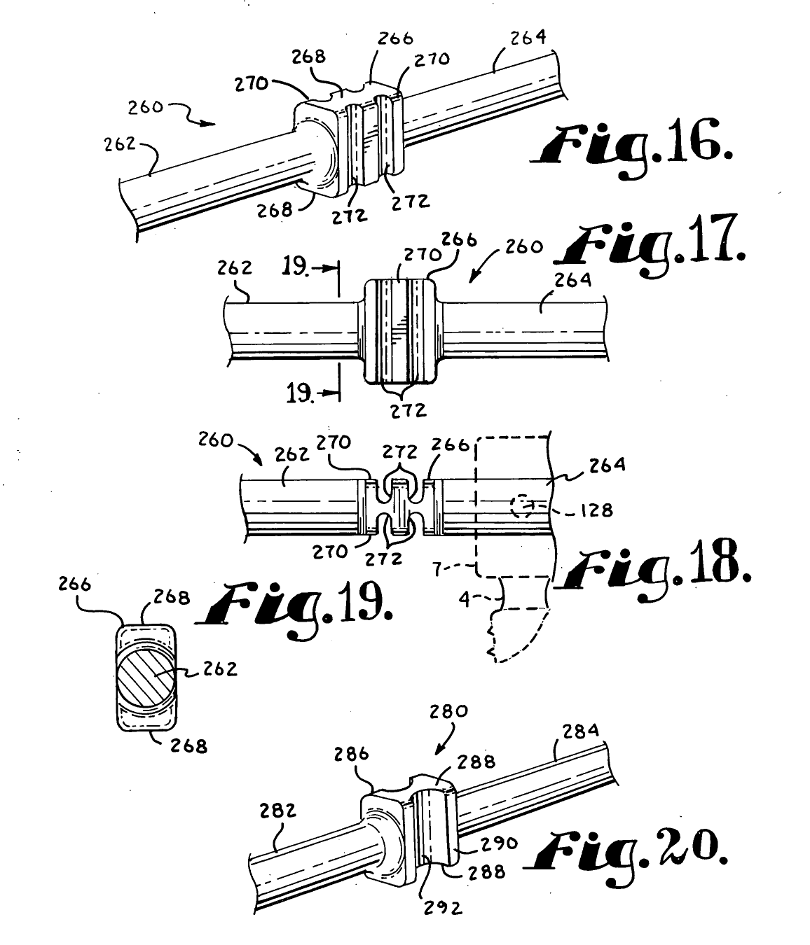

The illustrated second or replacement receiver 7 is substantially identical to the receiver 6 in form and function, constructed for engagement with the capture structure 10 as previously described herein with respect to the receiver 6, therefore the disclosure herein with respect to the receiver 6 is incorporated by reference with respect to the receiver 7. The receiver 7 differs from the receiver 6 in that the receiver 7 is sized to snugly receive the longitudinal connecting member or longitudinal member 20 that is of a different size diameter or width than the longitudinal connecting member 19. In the embodiment illustrated in FIG. 9, the receiver 7 has a U-shaped channel 160 having a lower seating surface 161, the channel 160 being wider than the channel 116 of the receiver 6. The channel 160 is sized and shaped to receive the longitudinal connecting member 20 that has a diameter or cross-sectional width larger than a diameter or cross-sectional width of the longitudinal connecting member 19.

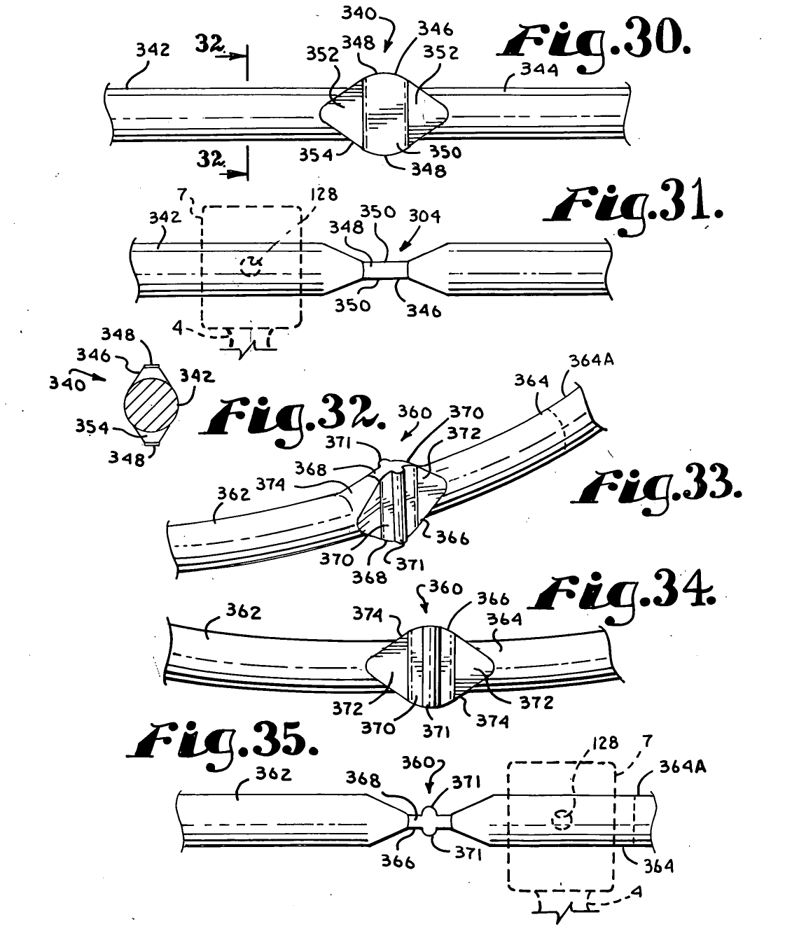

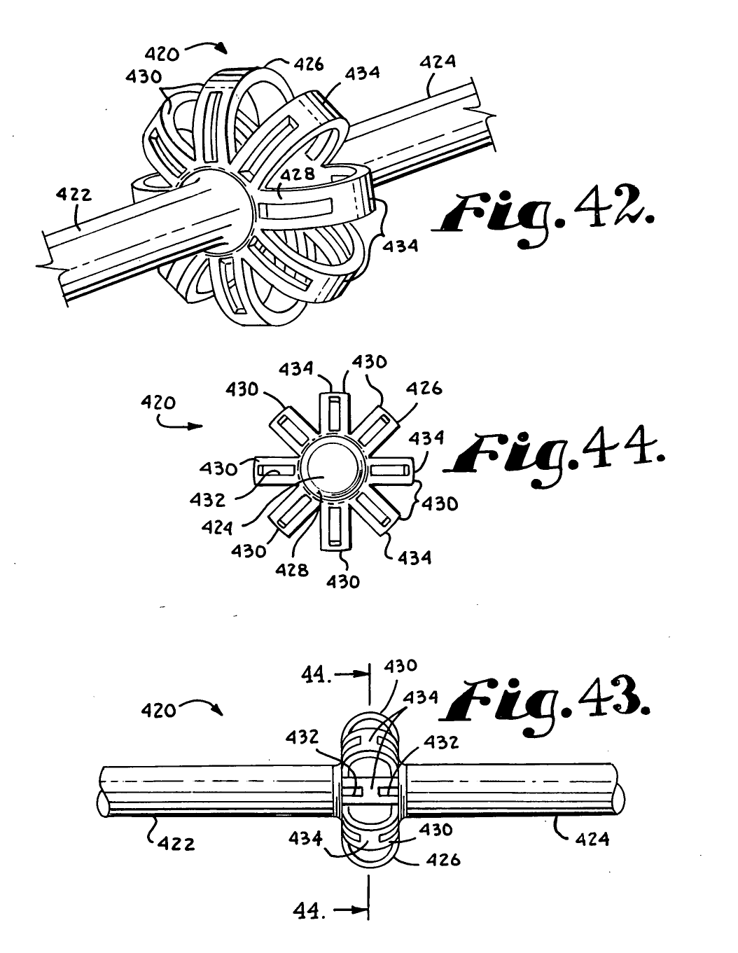

The elongate longitudinal connecting members or longitudinal members, such as the longitudinal connecting members 19 and 20 that are utilized with the assembly 1 can be any of a variety of implants utilized in reconstructive spinal surgery, but are typically elongate structures with substantially uniform cylindrical portions for placement within the receivers 6 and 7 respectively. The illustrated longitudinal connecting member 19 has a smaller diameter than a diameter of the illustrated longitudinal connecting member 20, providing an initial flexible structural connection between bone screw assemblies 1, that may then be replaced with a more rigid connection, if necessary, utilizing the replacement receiver 7 and the longitudinal connecting member 20, as will be described in greater detail below. As will also be described in greater detail below with respect to FIGS. 15-43, dynamic stabilization longitudinal connecting members according to the invention, such as the longitudinal connecting member 19 may be of a variety of configurations along a length thereof, some with uniform portions and others with non-uniform portions that are positioned between implanted spinal anchors, such as bone screws and hooks, providing structure for flexible, yet strong, dynamic stabilization medical implant assemblies.

The longitudinal connecting member portions that are received by the receiver 6 or 7 include cylindrical surfaces 162 or 163, respectively. The illustrated surfaces 162 and 163 are smooth but they could be textured. The longitudinal connecting members 19 and 20 are also preferably sized and shaped to snugly seat near the bottom of the U-shaped channels 116 and 160 of respective receivers 6 and 7, and, during normal operation, are positioned slightly above the bottom of the channels 116 and 160, respectively, near, but spaced from, respective lower surfaces 112 and 161. The longitudinal connecting member cylindrical surfaces 162 or 163 normally directly or abuttingly engage the shank top surface 78, as shown in FIGS. 8 and 10 and are biased against the dome shank top surface 78, consequently biasing the shank 4 downwardly in a direction toward the base of the receiver 6 or 7 when fully assembled with the longitudinal connecting member 19 or 10 and a closure member, such as the closure member 16. For this to occur, the shank top surface 78 must extend at least slightly into the space of the channel 116 or 160, above the respective surface 112 or 161 when the capture structure 10 is snugly seated in the lower part of the receiver cavity. The pressure placed on the capture structure 10 by the longitudinal connecting member 19 or 20 and a closure member may also cause a spreading or expansion of the capture structure 10, causing some interlocking or interdigitation between the guide and advancement structure 48 and the guide and advancement structure on the receiver 6 or 7. The shank 4 and the capture structure 10 are thereby locked or held in position relative to the receiver 6 or 7 by the longitudinal connecting member 19 or 20 respectively, firmly pushing downward on the shank domed surface 78.

With reference to FIGS. 1, 7 and 8, the closure structure or closure top 16 can be any of a variety of different types of closure structures for use in conjunction with the present invention with suitable mating structure on the upstanding arms 114 of the receiver 6. The closure top 16 is rotated between the spaced arms 114 and closes the top of the channel 116 to capture the longitudinal connecting member 19 therein. Likewise, a similar closure top (not shown) may be utilized to close the channel 160 of the receiver 10 to capture the longitudinal connecting member 20 therein.

The illustrated closure top 16 has a generally cylindrically shaped body 170, with a helically wound guide and advancement structure 172 that is sized, shaped and positioned so as to engage the guide and advancement structure 124 on the receiver arms 114 to provide for rotating advancement of the closure structure 16 into the receiver 6 when rotated clockwise and, in particular, to cover the top or upwardly open portion of the U-shaped channel 116 to capture the longitudinal connecting member 19, preferably without splaying of the arms 114. The body 170 further includes a base or bottom 174 having a pointed longitudinal connecting member engaging projection or point 175 extending or projecting axially beyond a lower rim 176. However, it is foreseen that the bottom could be flat and smooth and/or flat and knurled. The closure structure 16, with the projection 175 frictionally engaging and abrading the longitudinal connecting member surface 162, thereby applies pressure to the longitudinal connecting member 19 under torquing, so that the longitudinal connecting member 19 is urged downwardly against the shank domed surface 78 that extends into the channel 116. Downward biasing of the shank surface 78 operably produces a frictional engagement between the longitudinal connecting member 19 and the surface 78 and also urges the capture structure 10 toward the base 110 of the receiver 6, as will be described more fully below, so as to frictionally seat the capture structure buttress thread 48 and/or lower portion 72 against the threaded inner surface 52 of the receiver 6, also fixing the shank 4 and capture structure 10 in a selected, rigid position relative to the receiver 6.

The illustrated closure structure 16 further includes a substantially planar top surface 178 that has a centrally located, hexalobular internal driving feature 180 formed therein (sold under the trademark TORX), which is characterized by an aperture with a 6-point star-shaped pattern. It is foreseen that paired off-axis apertures, on-axis multi-lobular and other driving features or apertures, such as slotted, hex, tri-wing, spanner, and the like may also be utilized according to the invention. With reference to FIG. 7, a driving/torquing tool 181 having a cooperating hexalobular driving head is used to rotate and torque the closure structure 16. The tool 181 may also be utilized for removal of the closure structure 16, if necessary.

It is foreseen that a closure structure according to the invention may be equipped with a break-off feature or head, the closure structure sized and shaped to include a break-way region that breaks at a preselected torque that is designed to properly seat the closure structure in the receiver 6. Such a closure structure would include removal tool engagement structure, such as a pair of spaced apart apertures or bores, a countersunk hex-shaped aperture, a left hand threaded bore, or the like, fully accessible after the break-off head feature breaks away from a base of the closure structure.

In use, prior to the polyaxial bone screw assembly 1 being implanted in a vertebra according to the invention, the shank capture structure 10 is typically pre-loaded by insertion or bottom-loading into the receiver 6 through the opening 54 at the bottom end 56 of the receiver 6. The capture structure 10 is aligned with the receiver 6, with the axes A and B aligned so that the guide and advancement structure 48 of the capture structure 10 is inserted into and rotatingly mated with the guide and advancement structure 50 on the receiver 6. The shank 4 is rotated in a counter-clockwise direction to fully mate the structures 48 and 50, as shown in FIG. 3, and the counter-clockwise rotation is continued until the guide and advancement structure 48 disengages from the guide and advancement structure 50 and the capture structure 10 is fully disposed in the receiver cavity 136.

In the position shown in FIG. 4, the shank 4 is in slidable and rotatable engagement with the receiver 6, while the capture structure 10 is maintained in the receiver 6 with the shank body 8 in rotational relation with the receiver 6. The shank body 8 can be rotated through a substantial angular rotation relative to the receiver 6, both from side to side and from front to rear so as to substantially provide a universal or ball joint wherein the angle of rotation is restricted by the lower receiver opening 54.

With reference to FIGS. 5 and 6, the assembly 1 is then typically screwed into a bone, such as the vertebra 13, by rotation of the shank body 8 using the driving tool 31 that operably drives and rotates the shank 8 by engagement thereof with the hexagonally shaped extension head 76 of the shank 4. Preferably, when the driving tool 31 engages the head 76 during rotation of the driving tool 31, the outer portion 90 also engages the faces 84 and the bottom 91 of the tool 31 is fully seated upon and frictionally engages with the planar surface 82 disposed in the counter-sunk portion 80 of the capture structure 10. It is foreseen that in other embodiments according to the invention, the counter-sunk portion may be defined by more or fewer engaging surfaces, or the counter-sunk portion could be eliminated.

It is foreseen that in an alternative method according to the invention, the shank 4 is first implanted into the vertebra 13 by rotation of the shank 8 into the vertebra 13 using the driving tool 31 that operably drives and rotates the shank 8 by engagement thereof with the hexagonally shaped extension head 76 of the shank 4. As already described herein, when the driving tool 31 engages the head 76 during rotation of the driving tool 31, the outer portion 90 also engages the faces 84 and a bottom of the tool 31 is fully seated upon and frictionally engages with the planar surface 82 disposed in the counter-sunk portion 80 of the capture structure 10. It may be desirable to only partially implant the shank 8 into the vertebra 13, with the capture structure 10 extending proud to provide space for the attachment of the receiver 6 to the shank 4. The receiver 6 is then attached to the shank 4 by inserting the receiver 6 onto the capture structure with the axes A and B aligned and mating the thread 48 with the thread 50 by, rotating the receiver 6 in a clockwise direction. The head is then rotated until the thread 48 disengages with the thread 50 and the capture structure 10 is freely rotatably disposed in the head cavity 136. Then, the shank body the shank 4 can be further driven into the vertebra 13, if necessary, utilizing the driving tool 31 as already described herein. The remainder of the implant assembly includes elements that have been previously described.

With particular reference to FIGS. 5, 6 and 11, a procedure may begin by forming a relatively small incision in the skin for each bone screw shank 8 to be implanted. The incisions are stretched into a round shape with a circumference equal to or just slightly larger than the guide tools 130 and 131. The skin is relatively flexible and allows the surgeon to move the incision around relative to the spine to manipulate the various tools and implants, as required. The vertebra 13 may be pre-drilled with the small tap bore 105 to minimize stressing the bone and thereafter have the guide wire or pin 103 inserted therein to provide a guide for the placement and angle of the shank 4 with respect to the vertebra 13. A further bore (not shown) may be made with the guide pin 103 as a guide. Then, the assembly 1 is threaded onto the guide pin 103 utilizing the cannulation bore 92 by first threading the pin 103 into the bottom opening 96 and then out of the top opening 98. A receiver 6 is attached to a guide tool 130 or 131 and the shank body 8 is then driven into the vertebra 13, using the pin 103 as a placement guide.

With reference to FIGS. 7 and 11, the longitudinal connecting member 19 is eventually positioned within the head U-shaped channel 116 by inserting the longitudinal connecting member 19 diagonally through a skin incision near an end tool 130 so that a first longitudinal connecting member end passes through channels (not shown) in any intermediate guide tools 131 and into the channel (not shown) of the other end guide tool 130. Back muscle tissue separates easily here to allow the upper insertion of the longitudinal connecting member 19 and can be further separated by finger separation or cutting through one of the incisions if required. In a preferred method, once the longitudinal connecting member 19 is positioned within channels of the guide tools 130 and 131, the closure structure or top 16 is inserted into each of the tools 130 and 131 and advanced so as to bias or push against the longitudinal connecting member 19, pressing the longitudinal connecting member 19 to the bone screw receiver 6 and into the channel 116 by rotation of the closure top 16 between the arms 114. The closure structure 16 is rotated, utilizing the tool 181 in engagement with the driving feature or aperture 180 until an appropriate torque is achieved, for example 90 to 120 inch pounds, to urge the longitudinal connecting member 19 downwardly.

With reference to FIG. 8, the shank top domed surface 78, because it is rounded to approximately equally extend upward into the channel 116 approximately the same amount no matter what degree of rotation exists between the shank 8 and the receiver 6 and because the surface 78 is sized to extend upwardly into the U-shaped channel 116, the surface 78 is engaged by the longitudinal connecting member 19 and pushed downwardly toward the base 110 of the receiver 6 when the closure structure 16 biases downwardly toward and onto the longitudinal connecting member 19. Downward pressure on the shank 4 in turn urges the capture structure 10 base 34 and spherical crest surface 70 downward toward the receiver inner spherical surface 138 and crest surface 157. As the closure structure 16 presses against the longitudinal connecting member 19, the longitudinal connecting member 19 presses against the shank 4, and the capture structure 10 becomes frictionally and rigidly attached to the receiver 6. If the pressure is such that the capture structure 10 expands, a meshing and/or interlocking of the guide and advancement structure 48 and the guide and advancement structure 50 may occur, further fixing the shank body 8 in a desired angular configuration with respect to the receiver 6 and the longitudinal connecting member 19.

FIG. 8 illustrates the polyaxial bone screw assembly 1 with the longitudinal connecting member 19 and the closure structure 16 positioned in a vertebra 13. The axis A of the bone shank 8 is illustrated as not being coaxial with the axis B of the receiver 6 and the shank body 8 is fixed in this angular locked configuration. Other angular configurations can be achieved, as required during installation surgery due to positioning of the longitudinal connecting member 19 or the like.

According to a method of the invention, the bone screw assembly 1 and the longitudinal connecting member 19 are implanted as shown in FIGS. 8 and 11 to provide for a dynamic stabilization of a portion of the spine. In such a procedure, vertebrae are not prepared for fusion and the longitudinal connecting member 19 is not rigid, thus allowing for some flexible movement along the portion of the spine supported by the longitudinal connecting member 19. If, after time, further damage or weakness of the spine occurs, a method according to the invention allows for replacement of the flexible longitudinal connecting member 19 with the more rigid longitudinal connecting member 20, without removal of the bone screw shank 8 from the vertebra 13. With reference to FIGS. 9, 10 and 12, in such a procedure, partial disassembly is accomplished by using the driving tool 181 that is received in and mates with the driving feature 180 and then turned counterclockwise to rotate the closure structure 16 and reverse the advancement thereof in the receiver 6. Then, the longitudinal connecting member 19 may be removed in a percutaneous fashion in reverse order to the procedure described previously herein for assembly. The receiver 6 is then removed from the bone screw shank 4 by aligning the axis B of the receiver 6 with the axis A of the shank 4 and rotating the receiver in a counter-clockwise fashion with respect to the shank 4, the guide and advancement structure 50 aligned and rotatably mated with the guide and advancement structure 48 until the receiver 6 is detached from the shank 4.

If desired, selected vertebrae are abraded or otherwise prepared in a manner known in the art, including tissue removal, the addition of bone chip or other bone material, and/or bone growth promoting material, to result in fusion of the portion or portions of the spine being more rigidly fixed in place by the replacement receivers 7 and the longitudinal connecting member 20. Each replacement receiver 7 is then mounted on a capture structure 10, and rotated in a clockwise fashion, mating a guide and advancement structure on the inner surface of the receiver 7 (not shown) with the guide and advancement structure 48, the receiver 7 being rotated to fully mate the guide and advancement structures until the guide and advancement structure 48 is disengaged and the capture structure 10 is disposed in the receiver 7, the receiver 7 being freely rotatable with respect to the capture structure 10. The same procedure is followed along the spine to replace each receiver 6 with a receiver 7.

With reference to FIG. 12, the more rigid longitudinal connecting member 20 is installed in the receivers 7 similarly to what has been described previously herein with respect to the installation of the longitudinal connecting member 19 into the receivers 6. A closure structure (not shown), similar to the closure structure 16 is installed into each of the receivers 7, also as previously described herein.

With reference to FIGS. 13 and 14, a second embodiment of a bone screw assembly according to the invention, generally 201, includes a shank 204, a first receiver 206 and a second or replacement receiver 207. The shank 204 also includes a body 208 integral with an upper portion 209 having a spline capture structure 210. The assembly 201 further includes a retaining structure 211 adapted for fixed mating engagement with the spline capture structure 210 within the receiver 206 or 207. The spline capture connection between the capture structure 210 and the retaining structure 211 is described in detail in U.S. Pat. No. 6,716,214 and incorporated by reference herein. Also as described in the '214 patent, the retaining structure 211 includes a partially spherical surface 214 that is slidingly mateable with a cooperating inner surface of the receiver 206 or 207, allowing for a wide range of pivotal movement between the shank 204 and the receiver 206 or 207. In addition to what is described in U.S. Pat. No. 6,716,214, to provide a biologically active interface with the bone, an outer surface 216 of the shank body 208 that includes the thread is textured, coated, perforated or otherwise treated 218 as illustrated by a speckled surface on the drawing figures. The treatment 218 may include, but is not limited to a plasma spray coating, a hydroxyapatite (HA) coating, or other type of roughening, perforation or indentation in the surface 216, such as by sputtering, sand blasting or acid etching, that allows for bony on growth or ingrowth.

It is foreseen that other types of capture connections may also be used in bone screws according to the invention, including, but not limited to, conical, spherical, threaded, and frictional connections and retaining rings.

With reference to FIG. 13, the shank 204, the retaining structure 211 and the receiver 206 are typically assembled prior to implantation of the shank body 208 into a vertebra 213 by uploading the shank 204 into the receiver 206 and upwardly through the retaining structure 211 and then mating the spline capture structure 210 with the retaining structure 211 by rotating the capture structure 210 about a central axis of the shank 204 to about 60 degrees relative to the receiver, followed by downward movement, with splines of the capture structure 210 entering recesses in the retaining structure 211, as more fully described in U.S. Pat. No. 6,716,214. In an alternative method, the shank body 8 may be first implanted in the vertebra 13, followed by joining the receiver 6 to the shank 4 by mating the capture structure 210 with the retaining structure 211. Furthermore, as will be described in greater detail herein, the first receiver 6 may be removed from an implanted shank body 8 and the second receiver 7 joined to the shank 4 without the removal of the shank body 8 from the vertebra 13.

As illustrated in FIG. 14, the first receiver 206 is adapted to cooperate with a flexible longitudinal connecting member 219 of uniform diameter that is similar or identical to the longitudinal connecting member 19 described herein with respect to the assembly 1, and the second receiver 207 is adapted to cooperate with a more rigid longitudinal connecting member 220 (similar or identical to the longitudinal connecting member 20 of the assembly 1), the longitudinal connecting member 220 having a uniform diameter greater than a diameter of the flexible longitudinal connecting member 219. The shank upper portion 209 includes a curved or domed top surface 222 for contacting the longitudinal connecting member 219 or 220 in the same manner described previously herein with respect to the domed top 78 of the shank 4 and the longitudinal connecting members 19 or 20.

The longitudinal connecting member 219 is implanted into an assembly 201 having the first receiver 206 in a manner similar or identical to the implantation procedure previously described herein with respect to the longitudinal connecting member 19 and the assembly 1 having the receiver 6. It is foreseen that the shank 204 may or may not be cannulated and that the assembly 201 may further include a closure structure similar to the closure top 16 or other types of closure structure, for example, as described in U.S. Pat. No. 6,716,214, for advancement into the receiver 206 and biasing against the flexible longitudinal connecting member 219.

Similar to the procedure previously described herein with respect to the receiver 6 and the longitudinal connecting member 19, if, after time, further damage or weakness of the spine occurs, a method according to the invention allows for replacement of the flexible longitudinal connecting member 219 with the more rigid longitudinal connecting member 220, without removal of the bone screw shank 208 from the vertebra 213. In such a procedure, partial disassembly is accomplished by using a driving tool to remove the closure structures (not shown) from receivers 206, followed by removal of the longitudinal connecting member 219, preferably in a percutaneous fashion. The receiver 206 is then removed from the bone screw shank 204 by aligning the retaining structure/bone shank combination coaxially with the receiver 206 and then placing downward pressure on the retaining structure 211, causing the spline capture structure 210 to move upwardly toward the U-shaped channel of the receiver 206, disengaging the retaining structure 211 from the capture structure 210. The retaining structure 211 is then rotated about a central axis of the shank 204 about 60 degrees, aligning the splines of the capture structure 210 with axially aligned through-channels in the retaining structure 211, followed by upward movement of the receiver 206, with splines of the capture structure 210 entering the axial through-channels, allowing the receiver 206 and the retaining structure 211 to be disengaged and removed from the bone screw shank 204.

If desired, selected vertebrae are abraded or otherwise prepared in a manner known in the art, including but not limited to tissue removal, the addition of bone chip or other bone material, and/or bone growth promoting material to result in fusion of the portion or portions of the spine being more rigidly fixed in place by the replacement receivers 207 and the cooperating longitudinal connecting member 220. Each replacement receiver 207 with cooperating retaining structure 211 therein is then mounted on a capture structure 210 by placing the receiver 207 and aligned retaining structure 211 downwardly on the shank 204 until the capture structure 210 extends beyond the retaining structure 211. Then, the capture structure 210 is mated with the retaining structure 211 by rotating the retaining structure 211 about a central axis thereof about 60 degrees, followed by downward movement of the retaining structure, with splines of the capture structure 210 entering recesses in the retaining structure 211 as described in U.S. Pat. No. 6,716,214. The same procedure is followed along the spine to replace each receiver 206 with a receiver 207.

With reference to FIG. 12, the more rigid longitudinal connecting member 220 is installed, preferably percutaneously, in the receivers 207, similarly to what has been described previously herein with respect to the installation of the longitudinal connecting member 19 into the receivers 6. A closure structure (not shown), similar to the closure structure 16 or as described in U.S. Pat. No. 6,716,214, is installed into each of the receivers 7, also as previously described herein. It is noted that although the illustrated embodiments show replacing a smaller diameter rod with a larger diameter rod, in certain circumstances a larger diameter or width connecting member may be replaced by a member with a smaller width or cross-section. This may be desirable depending upon the materials chosen and other properties or geometries of the initially implanted connecting member and the replacement connector.

With reference to FIG. 15, in an alternative, non-uniform embodiment according to the invention, a longitudinal connecting member 250 may be used with the receiver 7, 207, or other receiver sized and shaped to receive the longitudinal connecting member 250, to provide a more flexible dynamic stabilization than that provided by the more rigid, uniform diameter longitudinal connecting members 20 or 220. The longitudinal connecting member 250 includes larger diameter sections 252 and smaller diameter sections 254, the sections 252 each having an axial length L of sufficient size to be fully received within a receiver, such as the receiver 7 or 207. The smaller diameter sections 254 may be of the same or varied lengths, and same or varied diameters, sized to extend between bone screw receivers of bone screws implanted to selected vertebra. In the embodiment illustrated in FIG. 15, there are four larger diameter sections 252 providing for the attachment of up to four bone screws or other bone anchors along a portion of a spine at varied distances corresponding to the various axial lengths of the smaller diameter sections 254. The longitudinal connecting member 250 may be made from a single piece of material and may include annular tapered portions 256, providing a diagonal bridge between each of the smaller diameter sections 254 and adjacent larger diameter sections 252. If circumstances require change-out to a more rigid, uniform diameter longitudinal connecting member, the longitudinal connecting member 250 may be removed from cooperating bone screws and a uniform diameter longitudinal connecting member, such as the longitudinal connecting member 20 or the longitudinal connecting member 220 may be inserted in the same receiver, making unnecessary the removal or change-out of receivers described previously herein with respect to the replacement of the longitudinal connecting member 19 with the longitudinal connecting member 20 or the longitudinal connecting member 219 with the longitudinal connecting member 220. The smaller diameter sections 254 that are designed to extend between bone screws may be sized to provide a more flexible, protected motion of the portions of the spine being repaired.

The longitudinal connecting member 250 also may be made of different materials (metal and non-metal) along the length thereof. For example, with respect to FIG. 15, one of the sections 254A may be made of a material exhibiting greater stiffness than the other sections 254 of similar (or varied) diameter. This would result in variable stiffness or flexibility along different segments or sections of the member 250. For example, a composite rod is possible, with the segment or section 254A made of a material with greater stiffness for promoting fusion (such as a metallic rod) and one or more connected or adjacent segments 254 of a different, more flexible material (such as a plastic or different metallic rod or non-uniform section as described in greater detail below with respect to FIGS. 16-43), could be used to provide protected movement of the one or more segments 254 that are connected to the segment 254A. The segments 254 and 254A may be attached to the sections 252 in a variety of ways, including, but not limited to fusing, welding, molding, casting, forging or other forms of adhering attachment to result in an integral relationship between the sections and/or segments. It is also foreseen that longitudinal connecting member portions of the same diameter, but made of different materials (different metals, different non-metals and combination of metal and non-metal) may be bonded, braided, molded, fused or otherwise adhered to one another, to result in an integral relationship therebetween. For example, a stiffer longitudinal connecting member portion made from a first material may be used for promoting fusion with the spine along a first selected length of the connecting member, while a more flexible, compressible and stretchable longitudinal connecting portion made from a second material, integral with the first portion, may be used to provide protected movement of the spine along a second selected length of the connecting member.

As previously discussed herein, a concern that arises in non-fusion dynamic stabilization procedures is the fatigue strength and thus the longevity of longitudinal connecting members and other structural members used in such procedures. In the apparatus and methods described thus far herein, the more flexible longitudinal connecting members 19, 219 and 250 may be changed out, when need arises, and replaced with identical replacement longitudinal connecting members 19, 219 or 250, respectively; with a more rigid longitudinal connecting member 20 or 220; or with a composite connecting member made from two or more different materials along a length thereof. Further embodiments according to the invention are shown in FIGS. 16-43 that include non-uniform portions that have an increased cross-sectional area. Increasing cross-sectional area increases fatigue strength and may further increase flexibility, at least in certain directions, thus providing some longevity to such longitudinal connecting members as well as increased protected motion of portions of the spine stabilized by such longitudinal connecting members in a non-fusion procedure. Such connecting members that include non-uniform portions, may also include portions made with different materials, e.g., a composite rod consisting, for example, of metallic and non-metallic materials.

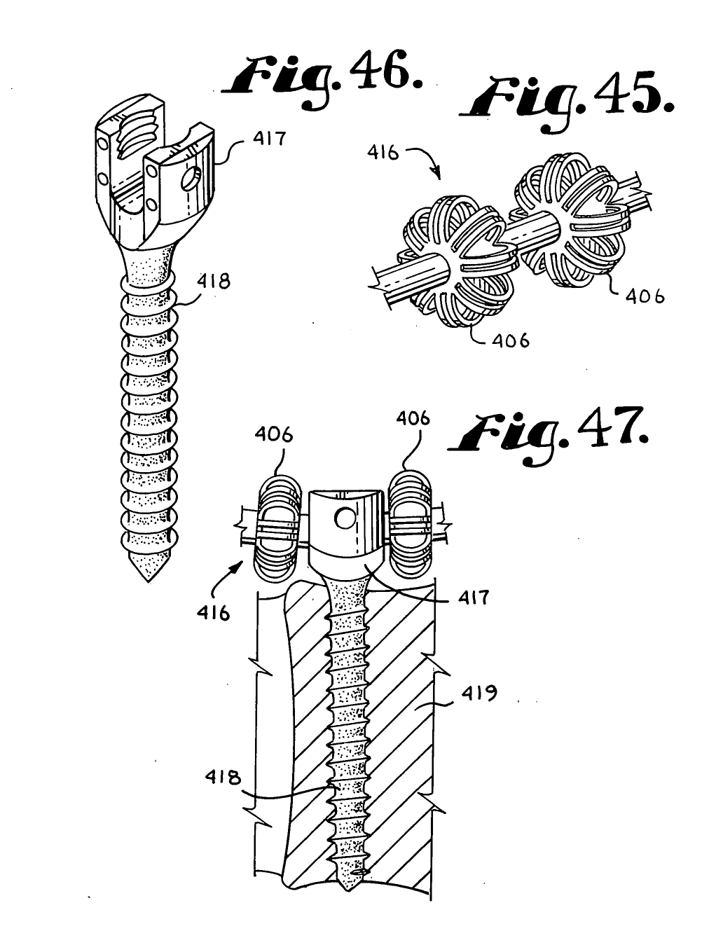

With reference to FIGS. 16-19, a second non-uniform longitudinal connecting member embodiment for use in apparatus and methods of the invention, generally 260 includes at least first and second uniform diameter cylindrical segments or portions 262 and 264, and at least one non-uniform portion or segment 266. The portions, 262, 264 and 266 are integral and substantially coaxial. In the illustrated embodiment the portion 262 has a diameter equal to a diameter of the portion 264; however, it is foreseen that portions 262 and 264 may have different diameters and cross-sectional shapes. The portions 262 and 264 are each receivable in a bone screw receiver, such as the receivers 6, 7, 206 and 207 previously described herein. The non-uniform portion 266 is designed for placement between bone screw receivers and is solid and substantially cuboid or parallelepiped in form, having a pair of substantially parallel surfaces 268 and a pair of substantially parallel grooved surfaces 270 disposed substantially perpendicular to the surfaces 268. A width or thickness of the portion 266 measured along an entire length of the surface 270 running between the parallel surfaces 268 is larger than the diameter of either of the uniform portions 262 and 264. A width or thickness of the portion 266 measured between the surfaces 270 is approximately equal to the diameter of the uniform portions 262 and 264. Each surface 270 includes a pair of open U-shaped grooves 272 running along an entire length thereof in a direction perpendicular to a longitudinal axis of the connecting member 260. A thinning of the portion 266 provided by the grooves 272, allows for increased flexing of the connecting member 260 at the non-uniform portion 266 as compared to the uniform portions 262 and 264. Also, because of the increased width measured between the surfaces 266, the non-uniform portion 266 is of increased cross-sectional area as compared to the uniform portions 262 and 264, providing for improved fatigue strength at the grooves 272. With reference to FIG. 18, a bone screw shank 4 and pivotally attached receiver 7 are shown schematically in phantom to provide a reference as to how the connecting member 260 is oriented with respect to such bone screw assembly when implanted in a vertebra. Although only one non-uniform portion 266 is shown in the drawing figures, it is foreseen that a plurality of portions 266 may be disposed on the connecting member 260, similar to what is shown, for example, with respect to a connecting member embodiment illustrated in FIG. 45 to be discussed in greater detail below.

With reference to FIGS. 20-22, a third non-uniform longitudinal connecting member embodiment for use in apparatus and methods of the invention, generally 280 includes at least first and second uniform diameter cylindrical segments or portions 282 and 284, and at least one non-uniform portion or segment 286. The portions 282, 284 and 286 are integral and substantially coaxial. In the illustrated embodiment the portion 282 has a diameter equal to a diameter of the portion 284; however, it is foreseen that portions 282 and 284 may have different diameters. The portions 282 and 284 are each receivable in a bone screw receiver, such as the receivers 6, 7, 206 and 207 previously described herein. The non-uniform portion 286 is designed for placement between bone screw receivers and is substantially solid and somewhat cuboid or parallelepiped in form, having a pair of substantially parallel surfaces 288 and a pair of substantially parallel surfaces 290 disposed substantially perpendicular to the surfaces 288 with a C- or U-shaped groove 292 carved into a substantial portion of each of the surfaces 290. A width or thickness of the portion 286 measured along an entire length of the surface 290 running between the parallel surfaces 288 is larger than the diameter of either of the uniform portions 282 and 284. A width or thickness of the portion 286 measured between the surfaces 290 is approximately equal to the diameter of the uniform portions 282 and 284. The groove 292 on each surface 290 runs along an entire length thereof in a direction perpendicular to a longitudinal axis of the connecting member 280. A thinning of the portion 286 caused by the grooves 292, allows for increased flexing of the connecting member 280 at the non-uniform portion 286 as compared to the uniform portions 282 and 284. Also, because of the increased width measured between the surfaces 288, the non-uniform portion 286 is of increased cross-sectional area as compared to the uniform portions 282 and 284, providing for improved fatigue strength at the grooves 292. With reference to FIG. 22, a bone screw shank 4 and pivotally attached receiver 7 are shown schematically in phantom to provide a reference as to how the connecting member 280 is oriented with respect to such bone screw assembly when implanted in a vertebra. Although only one non-uniform portion 286 is shown in the drawing figures, it is foreseen that a plurality of portions 286 may be disposed on the connecting member 280, similar to what is shown, for example, with respect to a connecting member embodiment illustrated in FIG. 45.

With reference to FIGS. 23-25, a fourth non-uniform longitudinal connecting member embodiment for use in apparatus and methods of the invention, generally 300 includes at least first and second uniform diameter cylindrical segments or portions 302 and 304, and at least one non-uniform portion or segment 306. The portions 302, 304 and 306 are integral and substantially coaxial. In the illustrated embodiment the portion 302 has a diameter equal to a diameter of the portion 304; however, it is foreseen that portions 302 and 304 may have different diameters. The portions 302 and 304 are each receivable in a bone screw receiver, such as the receivers 6, 7, 206 and 207 previously described herein. The non-uniform portion 306 is designed for placement between bone screw receivers and is somewhat cuboid or parallelepiped in form, having a pair of substantially parallel surfaces 308 and a pair of substantially parallel surfaces 310 disposed substantially perpendicular to the surfaces 308 with an aperture or through bore 312 extending between the surfaces 310, hollowing out a substantial part of the non-uniform portion 306. A width or thickness of the portion 306 measured along an entire length of the surface 310 running between the surfaces 308 is larger than the diameter of either of the uniform portions 282 and 284. A width or thickness of the portion 306 measured between the surfaces 310 is approximately equal to the diameter of the uniform portions 302 and 304. The through bore 312 runs parallel to the surfaces 308 in a direction perpendicular to a longitudinal axis of the connecting member 300. The hollowing out of the portion 306 caused by the bore 302, allows for compression and extension of the connecting member 300 at the non-uniform portion 306. Furthermore, at either side of the portion 306 are tapered necks 314, each having a diameter smaller than the diameter of the uniform portions 302 and 304. The tapered necks 314 provide space for deformation of the portion 306 when under compression and further provide for some flexibility or bending movement as compared to the uniform portions 302 and 304. With reference to FIG. 24, a bone screw shank 4 and pivotally attached receiver 7 are shown schematically in phantom to provide a reference as to how the connecting member 260 may be oriented with respect to such bone screw assembly when implanted in a vertebra. However, it is noted that because the portion 306 allows for compression and extension rather than bending, the portion 306 may be oriented in other directions also, for example, with the surfaces 310 rotated ninety degrees from what is shown in FIG. 24 with respect to the receiver 7. Although only one non-uniform portion 306 is shown in the drawing figures, it is foreseen that a plurality of portions 306 may be disposed on the connecting member 300, similar to what is shown, for example, with respect to a connecting member embodiment illustrated in FIG. 45.

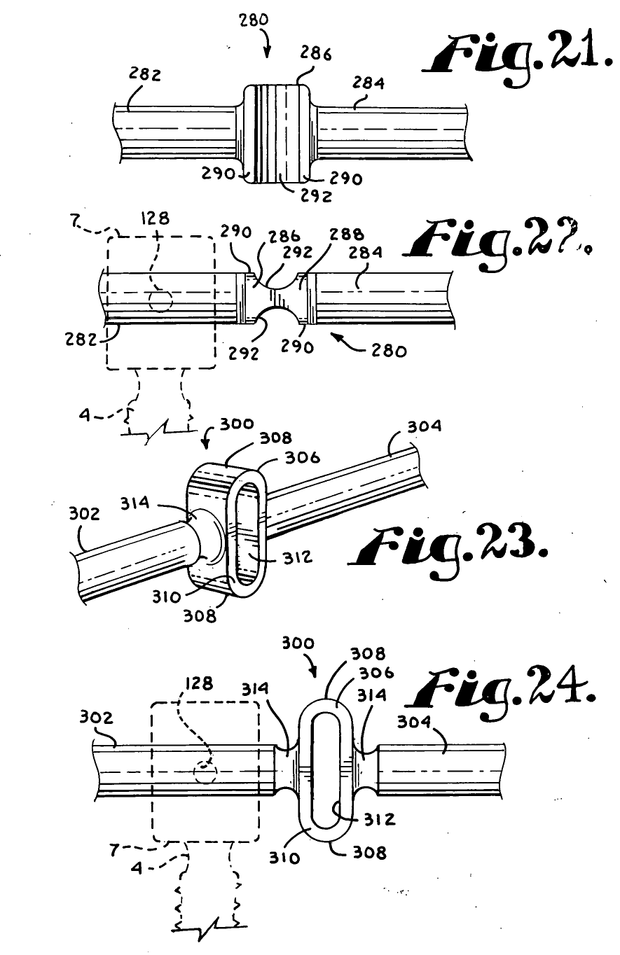

With reference to FIGS. 26-28, a fifth non-uniform longitudinal connecting member embodiment for use in apparatus and methods of the invention, generally 320 includes at least first and second uniform diameter cylindrical segments or portions 322 and 324, and at least one non-uniform portion or segment 326. The portions 322, 324 and 326 are integral and substantially coaxial. In the illustrated embodiment the portion 322 has a diameter equal to a diameter of the portion 324; however, it is foreseen that portions 322 and 324 may have different diameters. The portions 322 and 324 are each receivable in a bone screw receiver, such as the receivers 6, 7, 206 and 207 previously described herein. The non-uniform portion 326 is designed for placement between bone screw receivers and is substantially solid and cuboid or parallelepiped in form, having a pair of substantially parallel surfaces 328 and a pair of substantially parallel surfaces 330 disposed, substantially perpendicular to the surfaces 328. A width or thickness of the portion 326 measured along an entire length of the surface 330 running between the parallel surfaces 328 is larger than the diameter of either of the uniform portions 322 and 324. A width or thickness of the portion 326 measured between the surfaces 330 is smaller than the diameter of the uniform portions 322 and 324, thus providing for increased flexing of the connecting member 320 at the non-uniform portion 326 as compared to the uniform portions 322 and 324. Also, because of the increased width measured between the surfaces 328, the non-uniform portion 326 is of increased cross-sectional area as compared to the uniform portions 322 and 324, providing for improved fatigue strength as the portion 326 bends at the surfaces 330. With reference to FIG. 28, a bone screw shank 4 and pivotally attached receiver 7 are shown schematically in phantom to provide a reference as to how the connecting member 320 is oriented with respect to such bone screw assembly when implanted in a vertebra. Although only one non-uniform portion 326 is shown in the drawing figures, it is foreseen that a plurality of portions 326 may be disposed on the connecting member 320, similar to what is shown, for example, with respect to a connecting member embodiment illustrated in FIG. 45.

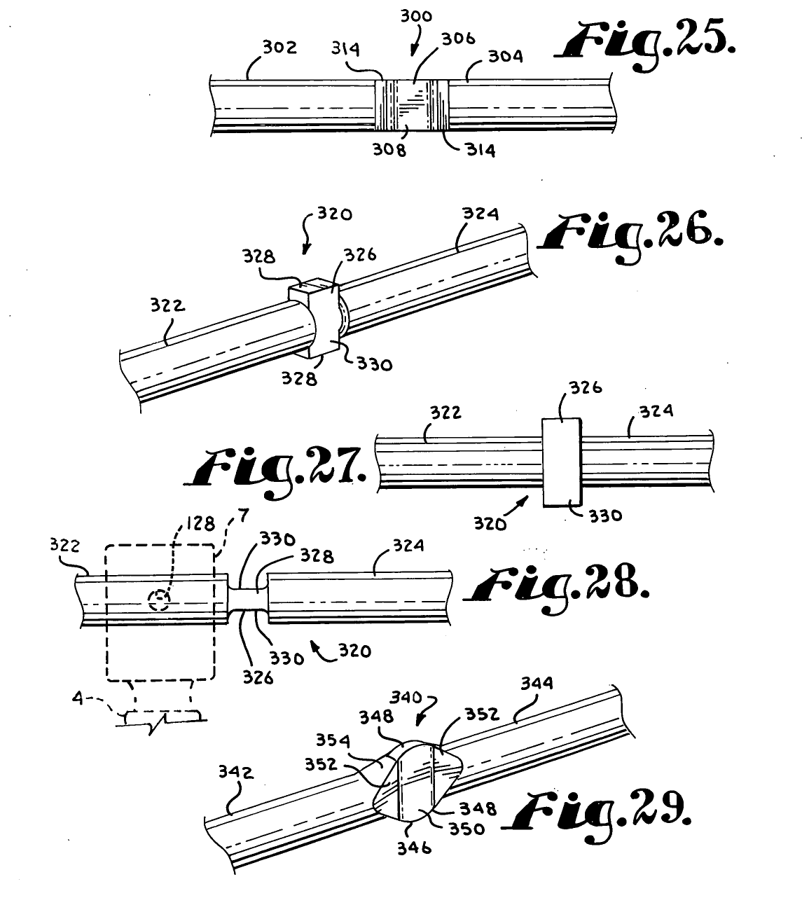

With reference to FIGS. 29-32, a sixth non-uniform longitudinal connecting member embodiment for use in apparatus and methods of the invention, generally 340 includes at least first and second uniform diameter cylindrical segments or portions 342 and 344, and at least one non-uniform portion or segment 346. The portions 342, 344 and 346 are integral and substantially coaxial. In the illustrated embodiment the portion 342 has a diameter equal to a diameter of the portion 344; however, it is foreseen that portions 342 and 344 may have different diameters. The portions 342 and 344 are each receivable in a bone screw receiver, such as the receivers 6, 7, 206 and 207 previously described herein. The non-uniform portion 346 is designed for placement between bone screw receivers and is substantially solid, having a pair of surfaces 348 that curve outwardly oppositely in one plane and a pair of substantially parallel surfaces 350 disposed substantially perpendicular to the surfaces 348. Relatively flat, sloping triangular surfaces 352 extend between the uniform portions 342 and 344 and the flat surfaces 350. Curved surfaces 352 slope outwardly from the cylindrical portions 342 and 344 to the curved surfaces 348. A width or thickness of the portion 346 measured along a length of the surface 350 running between the curved surfaces 348 is larger than the diameter of either of the uniform portions 342 and 344. A width or thickness of the portion 346 measured between the surfaces 350 is smaller than the diameter of the uniform portions 342 and 344, thus providing for increased flexing of the connecting member 340 at the non-uniform portion 346 as compared to the uniform portions 342 and 344. Also, because of the increased width measured between the surfaces 348, the non-uniform portion 346 is of increased cross-sectional area as compared to the uniform portions 342 and 344, providing for improved fatigue strength as the portion 346 bends at the surfaces 350. With reference to FIG. 31, a bone screw shank 4 and pivotally attached receiver 7 are shown schematically in phantom to provide a reference as to how the connecting member 340 is oriented with respect to such bone screw assembly when implanted in a vertebra. Although only one non-uniform portion 346 is shown in the drawing figures, it is foreseen that a plurality of portions 346 may be disposed on the connecting member 340, similar to what is shown, for example, with respect to a connecting member embodiment illustrated in FIG. 45.

With reference to FIGS. 33-35, a seventh non-uniform longitudinal connecting member embodiment for use in apparatus and methods of the invention, generally 360 includes at least first and second uniform diameter cylindrical segments or portions 362 and 364, and at least one non-uniform portion or segment 366. The portions 362, 364 and 366 are integral and substantially coaxial. It is noted that the longitudinal connecting member 360 is somewhat curved in form rather than following a straight longitudinal axis. Any or all of the connecting members described herein may be straight or curved, depending upon requirements and desired outcome of a particular surgical application. In the illustrated embodiment the portion 362 has a diameter equal to a diameter of the portion 364; however, it is foreseen that portions 362 and 364 may have different diameters. The portions 362 and 364 are each receivable in a bone screw receiver, such as the receivers 6, 7, 206 and 207 previously described herein. The non-uniform portion 366 is designed for placement between bone screw receivers and is substantially solid, having a pair of surfaces 368 that curve outwardly oppositely and a pair of substantially parallel surfaces 370 disposed substantially perpendicular to the surfaces 368. A pair of oppositely oriented ribs or ridges 371 are located centrally on the surfaces 370 and extend between the curved surfaces 368. Relatively flat, sloping triangular surfaces 372 extend between the uniform portions 362 and 364 and the flat surfaces 370. Curved surfaces 372 slope outwardly from the cylindrical portions 362 and 364 to the curved surfaces 368. A width or thickness of the portion 366 measured along a length of the surface 370 running between the curved surfaces 368 is larger than the diameter of either of the uniform portions 362 and 364. A width or thickness of the portion 366 measured between the surfaces 370 is smaller than the diameter of the uniform portions 362 and 364, thus providing for increased flexing of the connecting member 360 at the non-uniform portion 366 as compared to the uniform portions 362 and 364. Also, because of the increased width measured between the surfaces 368, the non-uniform portion 366 is of increased cross-sectional area as compared to the uniform portions 362 and 364, providing for improved fatigue strength as the portion 366 bends at the surfaces 360, the rib 371 also providing additional stability. With reference to FIG. 35, a bone screw shank 4 and pivotally attached receiver 7 are shown schematically in phantom to provide a reference as to how the connecting member 360 is oriented with respect to such bone screw assembly when implanted in a vertebra. Although only one non-uniform portion 366 is shown in the drawing figures, it is foreseen that a plurality of portions 366 may be disposed on the connecting member 360, similar to what is shown, for example, with respect to a connecting member embodiment illustrated in FIG. 45.

With further reference to FIG. 35, a longitudinal connecting member portion, length or segment 364A may also be made from a different material than a remainder length of the connecting member 360, resulting in a composite connecting member that varies in flexibility along the length of the entire longitudinal connecting member. For example, similar to what was described previously herein with respect to the segment 254A of the connecting member 250 illustrated in FIG. 15, the section, length or segment 364A may be made of a material of greater stiffness than an adjacent section 364 disposed on an opposite side of the bone screw shank 4, the section 364A being formed, fused, welded or otherwise adhered to be integral with the section 364, the stiffer section or segment 364A for promoting fusion along a length of the section or segment 364A between the illustrated implanted bone screw shank 4 and another bone screw (not shown) spaced from the shank 4 that is also attached to the section 364A. The segment 364A is preferably of a length sufficient to be received between a pair of bone anchors. In the illustrated embodiment, the sections or segments 362, 364, and 364A are of uniform cross-section, thus receivable in the same-sized bone anchor. Furthermore, each of the segments may be made from a substantially solid material.

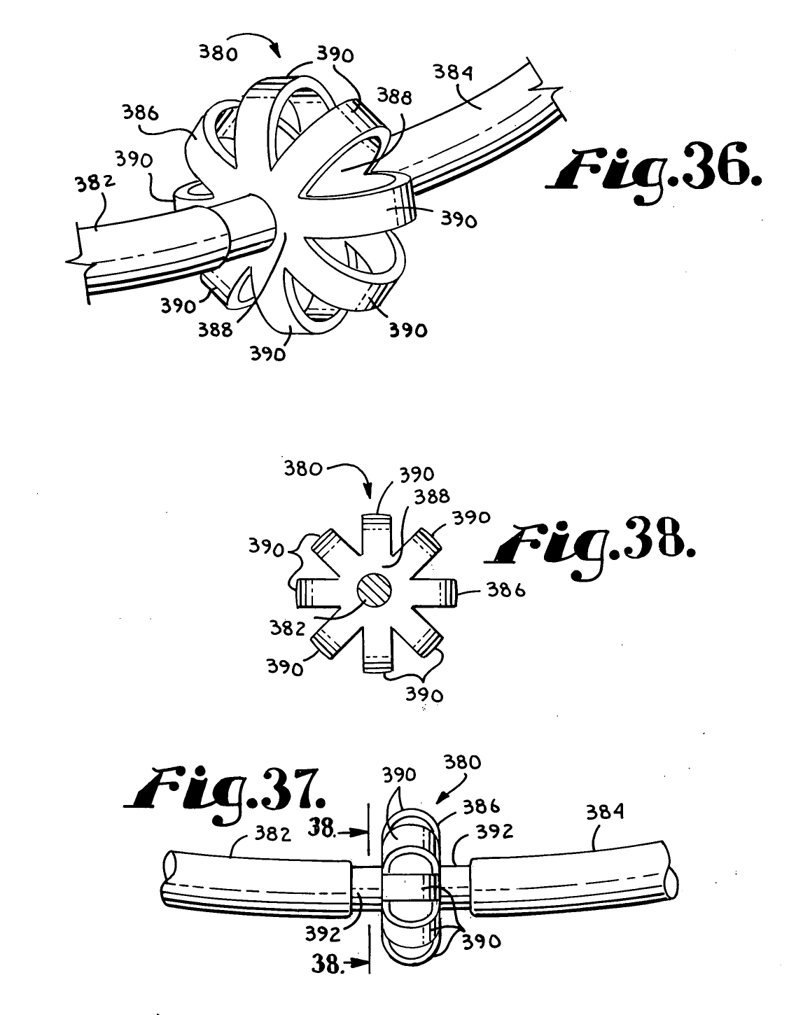

With reference to FIGS. 36-38, an eighth non-uniform longitudinal connecting member embodiment for use in apparatus and methods of the invention, generally 380 includes at least first and second uniform diameter cylindrical segments or portions 382 and 384, and at least one non-uniform cage-like portion or structure 386. The illustrated portions 382, 384 and 386 are integral and substantially coaxial. In the illustrated embodiment the portion 382 has a diameter equal to a diameter of the portion 384; however, it is foreseen that portions 382 and 384 may have different diameters. Furthermore, the portions 382, 384 and 386 may be made from different materials exhibiting different levels of stiffness or flexibility. The portions 382 and 384 are each receivable in a bone screw receiver, such as the receivers 6, 7, 206 and 207 previously described herein and similar to what is shown in FIG. 47. The non-uniform portion 386 is designed for placement between bone screw receivers and is substantially hollow, having a pair of substantially parallel sides 388, each side disposed substantially perpendicular to a longitudinal axis of the connecting member 380. Extending between and connecting the sides 388 are a plurality of strip-like segments or panels 390. Each of the segments 390 are integral with the sides 388, the portion 386 as well as the uniform portions 382 and 384 preferably being machined from a single piece of metal or non-metallic material. Although not shown in FIGS. 36-38, the structure 386 is hollowed out in a manner identical to that illustrated in FIGS. 41 and 44 for similar cage-like structures to be discussed in more detail below. The segments 390 are substantially U- or C-shaped and are uniformly spaced with respect to the longitudinal axis of the connecting member 380, forming a cage-like structure that is both compressible and expandable with openings between each of the segments 390. The compression and expansion occurs primarily at the U-shaped segments 390, but the sides 388 also move toward and away from one another in response to compression and tension. As previously stated, when in a neutral state or position (no compression and no expansion), the sides 388 of the structure 386 are substantially parallel. When the structure 386 is under tension and thus stretched, the structure 386 becomes somewhat ellipsoid in form. When compressed, the sides 388 are pressed toward one another, narrowing a width between legs of the U-shaped segments 390 and moving the segments 390 slightly radially outwardly. In the embodiment illustrated in FIGS. 36-38, there are eight segments 390, generally disposed at every 45 degrees as best shown in FIG. 38. At either side 388 of the cage-like structure 386 are tapered neck portions 392 that connect the structure 386 with the uniform portions 382 and 384. When the structure 386 is compressed due to movement of the uniform portions 382 and 384 toward one another, the neck portions move toward and into the structure 386, causing the sides 388 to move outwardly toward the uniform portions 382 and 384. The neck portions 392 of reduced diameter provide a relief or space for the sides 388 and the U-shaped segments 390 to move into, allowing for slightly increased movement of the connector 380 in an axial direction during compression. Although only one non-uniform portion 386 is shown in the drawing figures, it is foreseen that a plurality of portions 386 may be disposed on the connecting member 380, similar to what is shown, for example, with respect to a connecting member embodiment illustrated in FIG. 45. It is also noted that the non-uniform portion or portions of this and other longitudinal connecting member embodiments described herein may be sheathed, coated or otherwise covered by an inner or outer sleeve made from plastic or other flexible material so that bone and soft tissue growth does not occur between the portion segments that would inhibit the flexibility of the non-uniform portion.

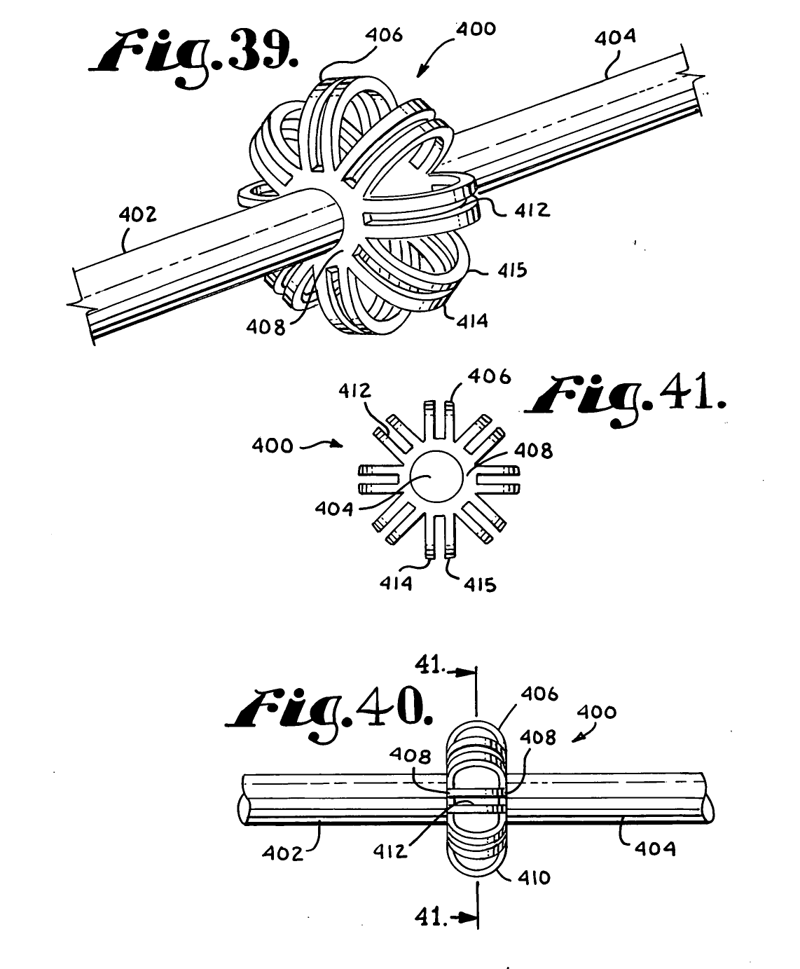

With reference to FIGS. 39-41, a ninth non-uniform longitudinal connecting member embodiment for use in apparatus and methods of the invention, generally 400 is substantially similar in structure and function to the connecting member 380 previously described herein and thus the discussion of the connecting member 380 is incorporated by reference herein with respect to the connecting member 400. The connecting member 400 includes at least first and second uniform diameter cylindrical segments or portions 402 and 404, and at least one non-uniform cage-like portion or structure 406. The portions 402, 404 and 406 are integral and substantially coaxial, with the portion 406 being hollow as illustrated in FIG. 41. The non-uniform portion 406 is designed for placement between bone screw receivers and is substantially similar to the portion 386, having a pair of substantially parallel sides 408, similar to the sides 388 and a plurality of U-shaped segments 410 connecting the sides 408, the segments 410 similar to the segments 390 with the exception that the segments 410 further include a slit or slot 412 disposed centrally in each segment 410 and extending from one side 408 to the other side 408. The slits 412 thus dividing each segment 410 into two strips 414 and 415, providing for increased compression and extension of the cage-like structure 406.

FIG. 45 illustrates two cage-like structures 406 mounted on a longitudinal connector, generally 416. FIG. 47 further shows the longitudinal connecting member 416 attached to a mono-axial or fixed bone screw 417. The bone screw 417 is discussed in detail in U.S. Pat. No. 6,726,687, incorporated by reference herein. In addition to what is described in U.S. Pat. No. 6,726,687, to provide a biologically active interface with the bone, the bone screw 417 includes a treated shank body 418 (illustrated as speckling on FIGS. 46 and 47). The shank body 418 treatment may include, but is not limited to a plasma spray coating, a hydroxyapatite (HA) coating, or other type of roughening, perforation or indentation in the surface 418, such as by sputtering, sand blasting or acid etching, that allows for bony on growth and ingrowth.

Although the longitudinal connecting member 416 is shown with the fixed bone screw 417, it is noted that the connector 416 and all other longitudinal connecting members 19, 20, 250, 260, 280, 300, 320, 340, 360, 380, 400 and 420 described in this application may be received in a variety of open bone screws, including, but not limited to, polyaxial, hinged and fixed bone screws as well as hooks and other types of bone anchors. It is further noted that all of the longitudinal connecting members described in this application may be made from metal or non-metallic materials as well as composites of such materials.

With reference to FIGS. 42-44, a tenth non-uniform longitudinal connecting member embodiment for use in apparatus and methods of the invention, generally 420 is substantially similar in structure and function to the connecting members 380 and 400 previously described herein and thus the discussion of the connecting members 380 and 400 are incorporated by reference herein with respect to the connecting member 420. The connecting member 420 includes at least first and second uniform diameter cylindrical segments or portions 422 and 424, and at least one non-uniform cage-like portion or structure 426. The portions 422, 424 and 426 are integral and substantially coaxial, with the portion 426 being hollow as illustrated in FIG. 44. The non-uniform portion 426 is designed for placement between bone screw receivers and is substantially similar to the portion 406, having a pair of substantially parallel sides 428, similar to the sides 408 and a plurality of U-shaped segments 430 connecting the sides 428, the segments 430 similar to the segments 390 and 410 with the exception that the segments 430 each include two slits or slots 432 one disposed in each side 428 and running into the segment 430, but not completely therethrough. Each slit 432 terminating at a central portion 434 of each segment 430, providing an area of additional strength when the segment 430 bends due to compressive forces.

It is to be understood that while certain forms of the present invention have been illustrated and described herein, it is not to be limited to the specific forms or arrangement of parts described and shown.

What is claimed and desired to be secured by Letters Patent is as follows:

1. A bone screw comprising:
a) a shank including a threaded lower portion adapted to be screwed into a bone and an upper portion with a capture structure; and
b) at least two interchangeable receivers adapted to receive and capture the shank upper portion capture structure, each of the receivers having a rod-receiving channel, wherein each of the channels is shaped to receive a rod having a different width.

2. A bone screw according to claim 1, wherein:
a) the shank upper portion capture structure is a helical guide and advancement structure adapted to cooperatively engage a complementary receiver guide and advancement structure such that the shank upper portion is rotatably received within the receiver.

3. A bone screw according to claim 1, further comprising:
a) a retaining structure adapted to cooperatively mate with the shank upper portion capture structure within the receiver.

4. A bone screw according to claim 3, wherein:
a) the shank upper portion capture structure includes at least one spline capture structure; and
b) the retaining structure includes a mating spline-engaging recess.

5. A bone screw comprising:
a) a shank including a threaded lower portion adapted to be screwed into a bone and an upper portion with a capture structure and a central upwardly projecting domed structure;
b) a retaining structure including a partially spherical outer surface; the retainer structure being releasably mateable with the capture structure of the shank upper portion; and
c) first and second interchangeable receivers; each of the receivers having a rod-receiving channel wherein the rod-receiving channels are shaped to received rods having different widths; the shank being received in the receiver and then held in the receiver by the retainer.

6. The bone screw according to claim 5, in combination with at least a first rod sized to be received in the rod-receiving channel of the first receiver.

7. The bone screw according to claim 6, in combination with at least a second rod sized to be received in the rod-receiving channel of the second receiver.

8. The bone screw according to claim 5, including:
a) a third interchangeable receiver with a rod-receiving channel shaped to receive a rod having a width different from the widths of the rods associated with the first and second interchangeable receivers.

9. The bone screw according to claim 5, each of the receivers including:
a) an inner cavity with a partially spherical inner surface cooperatively slidingly mateable with the outer surface of the retaining structure, so as to allow for a wide range of pivotal movement between the shank and the receiver; wherein
b) when the retaining structure is releasably mated with the capture structure of the shank upper portion, the domed structure extends into the rod-receiving channel so as to receive direct downward force from the rod received in the rod-receiving channel during locking of a position of the shank relative to the receiver.

10. The bone screw according to claim 5, wherein:
a) the shank domed structure includes an engagement surface on the top thereof.

11. The bone screw according to claim 5, wherein:
a) the shank domed structure includes a tool receiving surface adapted to receive a tool for rotating the shank into a bone.

12. The bone screw according to claim 11, in combination with at least a first rod sized to be received in the rod-receiving channel of the first receiver and a closure threadedly receivable in the first receiver so as to block the rod-receiving channel and apply downward force against the rod when locking the position of the rod relative to the receiver.

13. A bone anchor assembly comprising:
a) a shank including a threaded lower portion adapted to be screwed into a bone and an upper portion with a spline capture structure and a central upwardly projecting domed structure;
b) a retaining structure including a partially spherical outer surface and a spline engaging structure releasably fixedly mateable with the spline capture structure; and
c) a plurality of interchangeable receivers, each of the receivers including
i) an upper channel adapted to receive one of a plurality of rods differing from each other in at least one of structure, fabrication material, flexibility and elasticity, and
ii) an inner cavity with a partially spherical inner surface cooperatively slidingly mateable with the retaining structure outer surface so as to allow for a wide range of pivotal movement between the shank and the receiver; wherein
d) when the spline engaging structure is releasably mated with the spline capture structure, the domed structure extends into the upper channel so as to receive a direct downward force from the rod in the channel during locking of a position of the shank relative to the receiver.

14. The assembly according to claim 13, including a plurality of rods differing from each other in at least one of structure, fabrication material, flexibility and elasticity.

15. In a medical implant assembly including at least two bone attachment structures, the improvement comprising:
a longitudinal connecting member having an axis and further having first, second and third integral portions extending along the axis, the third portion being disposed between the first and second portions, the first and second portions being substantially uniform and each receivable in an open receiver of a bone attachment structure, the third portion having first and second substantially parallel axially spaced sides and a plurality of curved strips, each curved strip integral with both the first side and the second side at either end thereof, the third portion being both compressible and expandable along the axis.

16. In a medical implant assembly including at least two bone attachment structures, the improvement comprising:
a) a longitudinal connecting member having first, second and third integral and substantially coaxial portions, the third portion disposed between the first and second portions, the first and second portions being substantially uniform and receivable in an open receiver of a bone attachment structure, the third portion being hollow and substantially ellipsoid when under tension.

17. A medical implant kit comprising:
a) a polyaxial bone screw shank;
b) a first receiver having a first opening, the first receiver swivelably attachable to the shank and removable therefrom when the shank is implanted into a vertebra of a spine;
c) a first longitudinal connecting member closely receivable in the first opening of the first receiver, the first connecting member sized and shaped to allow protected motion of the spine;
d) a second receiver swivelably attachable to the shank when the shank is attached to the vertebra, the second receiver having a second opening of a different size than the first opening of the first receiver; and
e) a second longitudinal connecting member sized and shaped to be closely receivable in the second opening of the second receiver, the second longitudinal member having a different amount of flexibility than the first longitudinal member.

18. The kit of claim 17 wherein the bone screw shank includes at least one of
a) a surface roughening treatment; and
b) a coating to provide a bioactive interface between the bone attachment structure and a vertebra.

19. A surgical method comprising:
a) providing a polyaxial bone screw shank attachable to a first receiver;
b) implanting the shank and attached first receiver into a vertebra of a spine;
c) providing a first longitudinal connecting member sized and shaped to be closely receivable in the first receiver and to allow protected motion of the spine;
d) securing the first longitudinal connecting member in the first receiver; and
e) providing a second receiver attachable to the shank when the shank is implanted in the vertebra and sized and shaped to receive a second longitudinal connecting member, the second member having a different degree of flexibility than the first member.

20. The method of claim 19 comprising the subsequent steps of:
f) removing the first longitudinal connecting member from the first receiver;
h) replacing the first receiver with the second receiver without removing the bone screw shank from the vertebra; and
i) securing the second longitudinal connecting member to the second receiver.

21. A longitudinal connecting member comprising at least first and second elongate segments wherein each of the segments is sized and shaped to be adapted to be received between a pair of bone anchors; the first segment being comparatively stiff and the second segment being comparatively flexible in comparison to the first segment.

22. The longitudinal connecting member of claim 21 wherein at least a portion of the first segment is made from a material different from the second segment.
